(19) United States
(12) Patent Application Publication
(10) Pub. No.: US 2010/0056612 A1
(43) Pub. Date: Mar. 4, 2010

(54) MOLECULAR ENTITIES FOR BINDING, STABILIZATION AND CELLULAR DELIVERY OF CHARGED MOLECULES

(76) Inventors: Alexander Chucholowski, San Diego, CA (US); Thomas Hermann, Cardiff by the Sea, CA (US)

Correspondence Address: FOLEY & LARDNER LLP, P.O. BOX 80278, SAN DIEGO, CA, 92138-0278, US

(21) Appl. No.: 12/536,456

(22) Filed: Aug. 5, 2009

Related U.S. Application Data
(60) Provisional application No. 61/086,781, filed on Aug. 6, 2008.

(51) Int. Cl. A61K 31/7052; C07K 7/64; C07K 2/00; C07K 9/00; C08B 37/16; C12N 5/00; C07H 21/00; A61P 43/00

(52) U.S. Cl. 514/44 R; 530/317; 536/123.1; 530/300; 530/322; 536/103; 435/375; 536/23.1

(57) ABSTRACT

In accordance with the present invention, it has been discovered that the uptake of charged molecules into cells can be enhanced by noncovalently associating such molecules with molecular entities comprising an amphiphilic core with oppositely charged arms. The molecular entities form well defined stoichiometric complexes with charged molecules. Various compositions and methods for stabilizing anionic charged molecules and for enhancing the cellular uptake of any anionic charged molecules, e.g. double-stranded or hairpin nucleic acid, are provided.

### RELATED APPLICATION

This application claims benefit of priority from U.S. provisional application Ser. No. 61/086,781 filed Aug. 6, 2008 entitled “Molecular Entities for Binding, Stabilization and Cellular Delivery of Charged Molecules” which is incorporated by reference herein in its entirety.

### TECHNICAL FIELD

This invention relates to molecular entities for binding, stabilization and cellular delivery of charged molecules and for therapeutic treatment of diseases using same.

### BACKGROUND

The potential use of charged molecules such as polynucleotides as therapeutic agents has attracted great attention as a novel approach for treating severe and chronic diseases. However, polynucleotides have poor bioavailability and uptake into cells because polynucleotides do not readily permeate the cellular membrane due to charge repulsion between the negatively charged membrane and the high negative charge on the polynucleotide. In addition, polynucleotides are also highly susceptible to rapid nuclease degradation both inside and outside the cytoplasm; see examples from Geary et al, J. Pharmacol. Exp. Ther. 296:890-897 (2001).

One strategy to improve the structural stability of polynucleotides in vivo is to modify the phosphodiester backbone structure of the polynucleotides in efforts to reduce enzymatic susceptibility. Other strategies for addressing stability of polynucleotides and delivery thereof include condensation of cationic molecules (such as viral vectors) with polynucleotides and cationic delivery systems (such as lipid vesicles, lipid nanoparticles, polyethyleneimines and cyclodextrin-based polymers). However, concerns with intracellular vehicle fate and toxicity remain high.

### SUMMARY OF THE INVENTION

In accordance with the present invention, it has been discovered that the uptake of charged molecules into cells can be enhanced by noncovalently associating such molecules with molecular entities comprising an amphiphilic core with arms which bear a charge complementary to the charge of the charged molecule. For example, anionic charged molecules can be delivered employing molecular entities comprising cationic charged arms. Alternatively, cationic charged molecules can be delivered employing molecular entities comprising anionic charged arms. The molecular entities form well defined stoichiometric complexes with charged molecules. Various compositions and methods for stabilizing charged molecules and for enhancing the cellular uptake of any charged molecules, e.g. anionic charged molecules such as double-stranded or hairpin nucleic acids, are provided.

### BRIEF DESCRIPTION OF THE FIGURE

FIG. 1 compares the luciferase expression promoted by a test compound complexed with the luciferase knockdown sequence versus the luciferase expression promoted by the same test compound complexed with the scrambled knockdown sequence. In the figure, empty bars represent luc52/53tt (25 pmol), shaded bars represent 54/55 tt dicer (25 pmol) and blackened bars represent % knockdown.

### DETAILED DESCRIPTION OF INVENTION

In accordance with the present invention, there are provided molecular entities comprising an amphiphilic core and at least two charged arms covalently attached thereto, wherein said entities bind, stabilize and/or facilitate cellular delivery of charged molecules which bear a charge complementary to the charge of the charged arms. In some embodiments, the molecular entities bind, stabilize and/or facilitate cellular delivery of anionic charged molecules when positively charged arms are used. In other embodiments, the molecular entities bind, stabilize and or facilitate cellular delivery of cationic charged molecules when anionic charged arms are employed. In related embodiments, the amphiphilic cores employed in the practice of the present invention can be any amphiphilic molecules that have at least two attachment sites separated by a distance in the range of about 10-35 Angstroms for linkage of said arms to said core; preferably the distance is in the range of about 15-35 Angstroms.

The amphiphilic core may be a linearly extended structure or a macrocyclic structure which provides at least two attachment points for the charged arms. In some embodiments, the amphiphilic core may also interact with charged molecules, e.g., nucleic acid base pairs. The amphiphilic core is selected so as to interact with at least a portion of target molecules, e.g., solvent-exposed bases (purine and pyrimidine heterocycles) in nucleic acids, specifically such bases that are involved in base-pairing via hydrogen bonding. The amphiphilic core may be, on one side, a substantially flat or minimally convex surface which has relatively lower polarity (lower hydrophilicity) than the opposite side of the core, which has relatively higher polarity (higher hydrophilicity). This characteristic facilitates interaction with at least a portion of certain target molecules, e.g., solvent-exposed bases (e.g. purine or pyrimidine heterocycles) in nucleic acids, specifically such bases that are involved in base-pairing via hydrogen bonding. By interacting in said manner, the core surface of lower hydrophilicity shields the hydrophobic surface of the target molecules from interaction with other portions of the target molecules, and from unfavorable interactions with the solvent, which both potentially lead to aggregation and precipitation of the target molecules. Favorable interactions with the solvent, which might improve solubility of the complex, are achieved via the core surface of higher hydrophilicity, opposite to the surface of lower hydrophilicity.

Examples of linear core systems contemplated for use in the practice of the present invention, i.e., linear core systems with at least two attachment sites separated by a distance in the range of about 10-35 Angstroms, include, for example, substituted biphenyls (10 Angstrom distance between anchor points (A,B) at the para-positions), substituted biphenyl ethers (10 Angstrom distance between anchor points at the para-positions), triphenyl imidazoles and octaphenyl (35 Angstrom distance between anchor points for arms) as illustrated below. The distance between anchor points of the biphenyl system can be varied by employing a variety of linkers linking the two phenyl groups, e.g. an alkyl group, ether, amine, amide, aromatic, hetero-aromatic or a di-sulfide. Other suitable linear core systems such as bilirubin (15 Angstrom distance between anchor points), or the like are also suitable for use in the practice of the present invention.

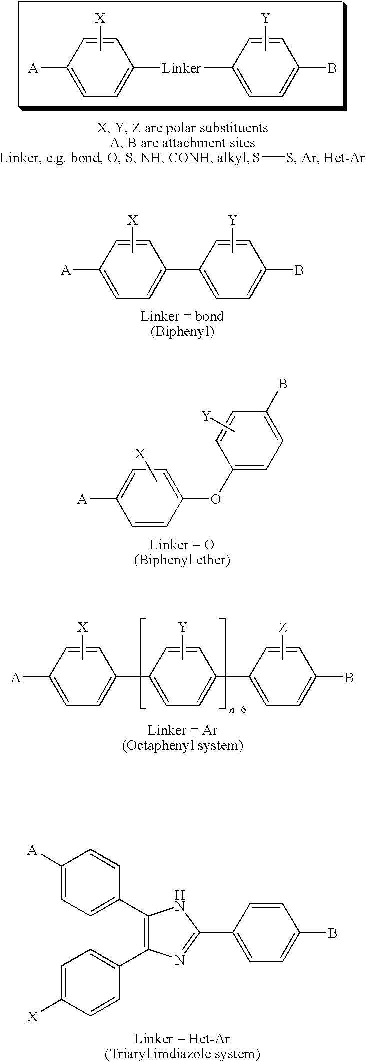

Exemplary macrocyclic molecules contemplated for use in the practice of the present invention as the amphiphilic core include cyclic peptides, cyclic oligosaccharides (e.g. cyclodextrins, cycloglucopyranosides), cyclic oligoethyleneglycols, substituted porphyrins, substituted corrins, substituted corroles, or the like (see examples illustrated below).

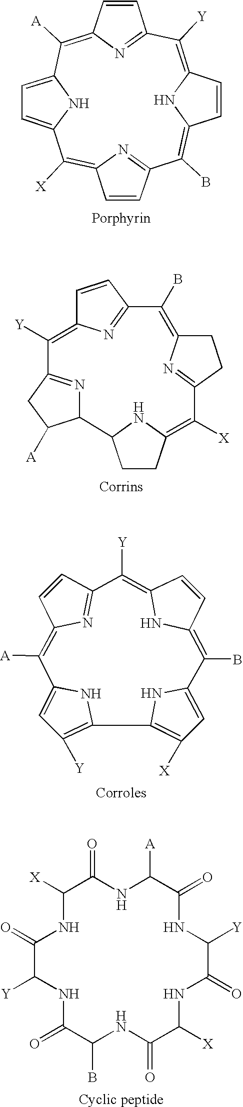

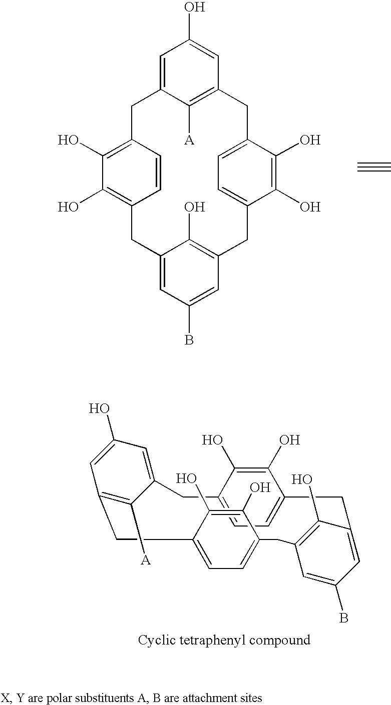

A person skilled in the art could readily identify other macrocyclic cores suitable for use in accordance with the present invention. For example, macrocyclic di-amines or di-amides as illustrated below may be used as a core to which charged arms may be attached at positions A and B, or at other possible positions deemed suitable for attachment.

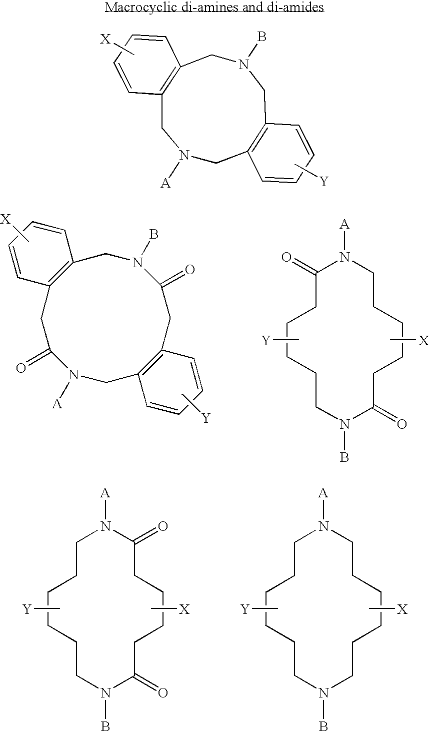

Alternatively cyclodextrins (CDs), a group of cyclic polysaccharides comprising six to eight naturally occurring D(+)-glucopyranose units in alpha-(1,4) linkage can be used as a core to which charged arms may be attached. The numbering of the carbon atoms of D(+)-glucopyranose units is illustrated below.

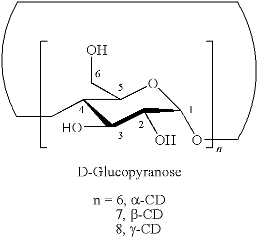

CDs are classified by the number of glucose units they contain: α-cyclodextrin has six glucose units; β-cyclodextrin has seven; and γ-cyclodextrin has eight. Each glucose unit is referred to as ring A, ring B, etc., as exemplified below for β-CD. The diameter of β-CD is measured to be around 5 Angstroms. In accordance with the present invention, the charged arms may be attached via the 6 positions of the at least A,C-, A,D- or A,E-rings of cyclodextrins.

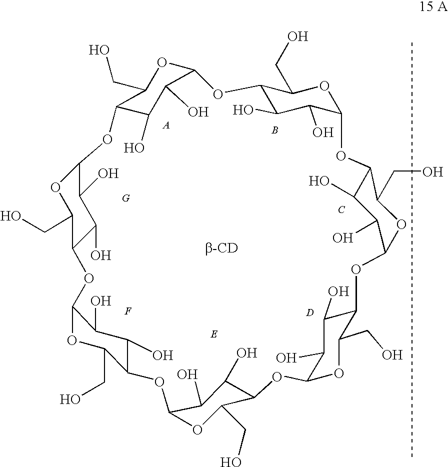

The three-dimensional architecture of CDs consists of cup-like shapes with relatively polar exteriors and nonpolar interiors. The resulting amphiphilic structure is thought to be able to imbibe hydrophobic compounds to form host-guest complexes. According to both in vitro and in vivo studies, CDs, especially alkylated CD derivatives, may have enhancer activity on transport through cell membranes. For example, Agrawal et al. (U.S. Pat. No. 5,691,316) describes a composition including an oligonucleotide complexed with a CD to achieve enhancing cellular uptake of oligonucleotide.

Other cyclic oligosaccharides, such as β-1,6-thio-linked cycloglucopyranosides or cyclic tetrasaccharide, cyclo{-6)-α-D-Glcp-(1,3)-α-D-Glcp-(1,6)-α-D-Glcp-(1,3)-α-D-Glcp-1-}, and the like are also suitable for use as a core in accordance with the present invention.

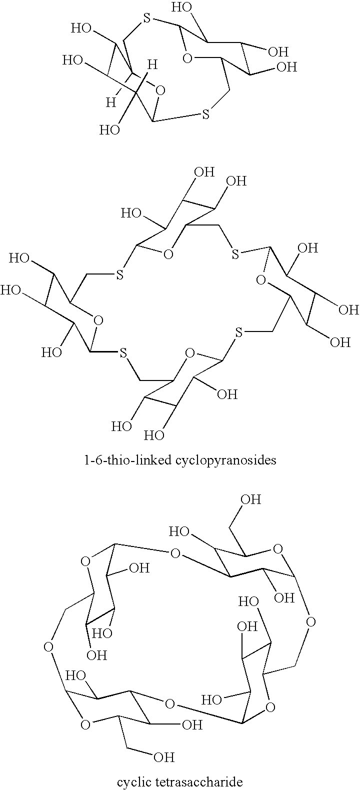

In some embodiments of the present invention, the macrocyclic molecules may be oligosaccharides other than cyclodextrins. In certain embodiments, the macrocyclic molecules maybe cyclic peptides or cyclic oligoethyleneglycols.

In some embodiments, the negatively charged arms comprise a plurality of residues selected from carboxylic acids, sulfonic acids, sulfuric acids, phosophonic acids, phosphoric acids, or combinations thereof. In related embodiments, one or both of the negatively charged arms further comprises neutral and/or polar functional groups. In related embodiments, each negatively charged arm may comprise a plurality of reactive units selected from the group consisting of alpha-amino acids, beta-amino acids, gamma-amino acids, anionically functionalized monosaccharides, anionically functionalized ethylene glycols, and combinations thereof. In preferred embodiments, each anionic charged arm may be an oligomer selected from the group consisting of oligopeptide, oligoamide, anionically functionalized oligoether, anionically functionalized oligosaccharide, and combinations thereof.

In some embodiments, the positively charged arms comprise a plurality of residues selected from amines, guanidines, amidines, N-containing heterocycles, or combinations thereof. In related embodiments, one or both of the cationic arms may further comprise neutral and/or polar functional groups, for example, PEGs or fatty acids (either as part of the backbone of the cationic arms or as an substituent thereon). In related embodiments, each positively charged arm may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. In related embodiments, one or both of the cationic arms may further comprise neutral and/or polar functional groups, for example, PEGs or fatty acids (either as part of the backbone of the cationic arms or as an substituent thereon). In preferred embodiments, each positively charged arm may comprise oligomer(s) independently selected from the group consisting of oligopeptide, oligoamide, cationically functionalized oligoether, cationically functionalized oligosaccharide, oligoamine, oligoethyleneimine, and combinations thereof. The oligomers may be oligopeptides where all the amino acid residues of the oligopeptide are capable of forming positive charges. In yet other embodiments, the length of the contiguous backbone of each positively charged arm is about 12 to about 200 Angstroms; preferably about 12 to about 100 Angstroms. For example, the positively charged arms may be oligopeptides comprising 3 to 50 amino acids (approximately about 12 to about 200 Angstroms); preferably 3 to 40 amino acids; more preferably 6 to 30 amino acids.

As used herein, the term “about” refers to ±10% of a given measurement.

As used herein, the term “amino acids” include the (D) and (L) stereoisomers of such amino acids when the structure of the amino acid admits stereoisomeric forms. The configuration of the amino acids and amino acid residues herein are designated by the appropriate symbols (D), (L) or (DL), furthermore when the configuration is not designated the amino acid or residue can have the configuration (D), (L) or (DL).

As used herein, the term “anionic functional monosaccharides” may include any carboxylic acid-containing monosaccharide such as uronic acid, aldaric acid, aldonic acid, ketoaldonic acid, N-acetyl-neuraminic acid and sialic acid. It may also include any natural or unnatural derivatized monosaccharides containing one or more functional groups that can form negative charge, e.g. carboxylic, sulfonic, sulfuric, phosophonic, or phosphoric acid containing groups.

As used herein, the term “anionically functionalized oligosaccharide” refers to an oligosaccharide comprising one or more “anionically functionalized monosaccharides.”

As used herein, the term “anionically functionalized ethylene glycols” may include any substituted ethylene glycols where the substituents comprise functional groups that can form anionic charge, e.g. carboxylic, sulfonic, sulfuric, phosophonic and phosphoric acid containing groups.

As used herein, the term “anionically functionalized oligoether” may include any substituted oligoether where the substituents comprise functional groups that can form anionic charge, e.g. carboxylic, sulfonic, sulfuric, phosophonic and phosphoric acid containing groups.

As used herein, the term “cationically functional monosaccharides” may include any amine-containing monosaccharide such as glucosamine, galactosamine and 2-amino-sialic acid. It may also include any natural or unnatural derivatized monosaccharides containing one or more functional groups that can form positive charge, e.g. amine and phosphorus containing groups.

As used herein, the term “cationically functionalized oligosaccharide” refers to an oligosaccharide comprising one or more “cationically functionalized monosaccharides.”

As used herein, the term “cationically functionalized ethylene glycols” may include any substituted ethylene glycols where the substituents comprise functional groups that can form positive charge, e.g. amine and phosphorus containing groups.

As used herein, the term “cationically functionalized oligoether” may include any substituted oligoether where the substituents comprise functional groups that can form positive charge, e.g. amine and phosphorus containing groups.

In some embodiments, invention entities may further comprise a bio-recognition molecule. In certain aspects, the bio-recognition molecule could be covalently linked or non-covalently linked to the molecular entities. The bio-recognition molecules optionally incorporated into the molecular entities may be any molecules such as oligopeptides or oligosaccharides that are involved in a large range of biological processes including cell attachment, cell penetration and cell recognition so as to promote binding of, recognition of or cell penetration of such molecules. Examples of such bio-recognition molecules include peptidyl-cyclodextrins which can be found in Pean et al. J. Chem. Soc. Perkin Trans. 2, 2000, 853-863. Exemplary molecules include TAT peptides (Transacting Activator of Transcription peptide), linear or cyclic RGD (Arg-Gly-Asp) peptides or RGD peptide mimetics.

In some embodiments, the charged arms are represented by formula I:

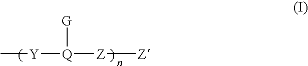

wherein:
G is hydrogen, cationically or anionically functionalized side chain;
Y is independently a covalent bond, O, NR¹, C(═X) or S(═O)ₘ;
Z is independently a covalent bond, O, NR¹, C(═X) or S(═O)ₘ;
Q is independently selected from the group consisting of (CH)ₚ, ethylene imine, ethylene glycol or monosaccharide;
Z′ is R¹, OR¹, NR¹ or SR¹;
R¹ is hydrogen or lower alkyl;
X is O, S or NR¹;
n is an integer ranging from 3 to 50;
m is 0, 1, or 2; and
p is 1, 2, 3, or 4.
   In preferred embodiments, G is a cationically functionalized side chain with a length of about 3 to about 12 Angstroms comprising functional groups that form one or more positive charges, e.g. amine or phosphorus-containing functional groups. G may be —CH₂—(CH₂)ₙ—W; wherein W is amino, amidino, guanidinyl, imidazolyl or phosphorus containing group. Examples of such side chain may include lysine side chain, arginine side chain, histidine side chain, ornithine side chain, and the like. A skilled artisan would readily realize when n=1, —CH₂—(CH₂)ₙ—W is about 3 Angstroms in length and when n=10. —CH₂—(CH₂)ₙ—W, is about 12 Angstroms in length. W may independently be further derivatized with PEGs, fatty acids or bio-recognition molecules, so long as the arm is positively charged. The skilled artisan could also readily identify other side chains suitable for use in the practice of the present invention.

In other embodiments, G is an anionically functionalized side chain with a length of about 3 to 12 Angstroms comprising functional groups that form one or more negative charges, e.g. carboxylic, sulfonic, sulfuric, phosophonic or phosphoric acid containing functional groups. G may be —CH₂—(CH₂)ₙ—W′; wherein W′ is carboxylic, sulfonic, sulfuric, phosophonic or phosphoric acid containing group. Examples of such side chain may include aspartic acid side chain, glutamine acid side chain, and the like. The skilled artisan would readily realize when n=1, —CH₂—(CH₂)ₙ—W′ is about 3 Angstroms in length and when n=10, —CH₂—(CH₂)ₙ—W′, is about 12 Angstroms in length. W′ may independently be derivatized with PEGs, fatty acids or bio-recognition molecules, so long as the arm is negatively charged. The skilled artisan could also readily identify other side chains suitable for use in the practice of the present invention.

In accordance with the present invention, the length of the contiguous backbone of the charged arms is selected so as to correspond to the specific oppositely charged molecules which are intended to interact with the molecular entities. In some embodiments, the length of the contiguous backbone of each of the charged arms is about 12 to about 200 Angstroms; preferably about 12 to about 160 Angstroms; more preferably about 12 to about 120 Angstroms; most preferably about 12 to about 80 Angstroms. For example, when the amphiphilic core provides an anchor for one end of a charged molecule (such as a nucleic acid strand), and assuming that the closest distance between two stacked nucleotides is around 2.5 Angstroms, the lower limit of about 12 Angstroms for the arm length corresponds to a nucleic acid of about 5 nucleotides while the upper limit of about 200 Angstroms corresponds to a nucleic acid of about 80 nucleotides.

In some embodiments, the anionic charged molecules may be a double-stranded or hairpin nucleic acid. In other embodiments, the anionic charged molecules may be selected from the group consisting of single-stranded DNA, double-stranded DNA, single-stranded RNA, double-stranded RNA, and oligonucleotide comprising non-natural monomers including 2′-methoxy or 2′-fluoro-modified nucleotides with ribo- or arabino-stereochemistry at the 2′-position, or thio-substituted phosphate groups or the like. The single-stranded RNA may be mRNA or miRNA. The double-stranded RNA may be siRNA.

As used herein, the term “nucleic acids” are oligonucleotides consisting of deoxyribonucleic acid (DNA) or ribonucleic acid (RNA), or chimeric oligonucleotides, containing DNA and RNA, or oligonucleotide strands containing non-natural monomers, including but not limited to 2′-methoxy or 2′-fluoro-modified nucleotides with ribo- or arabino-stereochemistry at the 2′-position, or thio-substituted phosphate groups. Nucleic acids contemplated for use in the practice of the present invention may also include conjugated nucleic acids where nucleic acids conjugate to protein, polypeptide or any organic molecules.

As used herein, “double-stranded nucleic acids (hybrids)” are formed from two individual oligonucleotide strands of substantially identical length and complete or near-complete sequence complementarity (“blunt end hybrids”) or offset sequence complementarity (“symmetrical overhang hybrids”, not necessarily implying sequence identity of the overhanging monomers), or from strands of different lengths and complete or offset sequence complementarity (“overhang hybrids”). In symmetrical overhang hybrids, preferred number of the non-hybridized overhang nucleotides is between 1-10; more preferred is between 1-4; most preferred is between 1-2.

As used herein, “sequence complementarity” is defined as the ability of monomers in two oligonucleotides to form base pairs between one nucleotide in one strand and another nucleotide in the second strand by formation of one or more hydrogen bonds between the monomers in the base pair.

As used herein, “complete sequence complementarity” means that each residue in a consecutive stretch of monomers in two oligonucleotides participates in base pair formation.

As used herein, “near-complete sequence complementarity” means that a consecutive stretch of base pairs is disrupted by no greater than one unpaired nucleotide per 3 consecutive monomers involved in base pairing. Preferably, base pairing refers to base pairs between monomers that follow the Watson-Crick rule (adenine-thymine, A-T; adenine-uracil, A-U; guanine-cytosine, G-C) or form a wobble pair (guanine-uracil, G-U).

As used herein, “hairpin nucleic acids” are formed from a single oligonucleotide strand that has complete or near-complete sequence complementarity or offset sequence complementarity between stretches of monomers within the 5′ and 3′ region such that, upon formation of intra-oligonucleotide base pairs, a hairpin structure is formed that consists of a double-stranded (hybridized) domain and a loop domain which contains nucleotides that do not participate in pairing according to the Watson-Crick rule. Preferred length of hairpin oligonucleotides is between 15-70 monomers (nucleotides); more preferred length is between 18-55 monomers; even more preferred length is between 20-35 monomers; most preferred length is between 21-23 monomers. A skilled artisan will realize nucleotides at the extreme 5′ and 3′ termini of the hairpin may but do not have to participate in base pairing.

The terms “polynucleotide” and “nucleic acid molecule” are used broadly herein to refer to a sequence of two or more deoxyribonucleotides, ribonucleotides or analogs thereof that are linked together by a phosphodiester bond or other known linkages. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. The terms also are used herein to include naturally occurring nucleic acid molecules, which can be isolated from a cell using recombinant DNA methods, as well as synthetic molecules, which can be prepared, for example, by methods of chemical synthesis or by enzymatic methods such as by PCR. The term “recombinant” is used herein to refer to a nucleic acid molecule that is manipulated outside of a cell, including, for example, a polynucleotide encoding an siRNA specific for a histone H4 gene operatively linked to a promoter. Preferred length of oligonucleotides in double-stranded nucleic acids is between 15-60 monomers; more preferred length is between 15-45 monomers; even more preferred length is between 19-30 monomers; most preferred length is between 21-27 monomers.

The charged arms may be directly linked to the amphiphilic core via procedures known in the art. For example, oligopeptide arms may be directly attached to the 6 hydroxyl groups of beta-cyclodextrin via an ester linkage. On the other hand, the arms may be indirectly linked to the amphiphilic core via other suitable linkers. In some embodiments, each linker of the entities is independently selected from the group consisting of a disulfide linkage, a protected disulfide linkage, an ether linkage, a thioether linkage, a sulfoxide linkage, a sulfonate linkage, an amine linkage, a hydrazone linkage, a sulfonamide linkage, an urea linkage, an ester linkage, an amide linkage, a carbamate linkage, a dithiocarbamate linkage, and the like, as well as combinations thereof.

Linkers with more than one orientation for attachment to the amphiphilic core can be employed in all possible orientations for attachment. For example, an ester linkage may be oriented as —OC(O)— or —C(O)O—; a sulfonate linkage may be oriented —OS(O)₂— or —S(O)₂O—; a thiocarbamate linkage may be oriented —OC(S)NH— or —NHC(S)O—. A skilled artisan would readily recognize other suitable linkers for attachment of each charged arm.

In some embodiments, invention entities further comprise a bio-recognition molecule. In certain aspects, the bio-recognition molecule could be covalently linked or non-covalently linked to the molecular entities. The bio-recognition molecules optionally incorporated into the molecular entities may be any molecules such as oligopeptides or oligosaccharides that are involved in a large range of biological processes including cell attachment, cell penetration and cell recognition so as to promote binding of, recognition of or cell penetration of such molecules. Examples of such bio-recognition molecules include peptidyl-cyclodextrins which can be found in Pean et al. J. Chem. Soc. Perkin Trans. 2, 2000, 853-863. Exemplary molecules include TAT peptides (Transacting Activator of Transcription peptide), linear or cyclic RGD (Arg-Gly-Asp) peptides or RGD peptide mimetics.

In other embodiments, the present invention provides methods for delivering a charged molecule to a cell, said method comprising:
a) binding non-covalently a molecular entity of as described herein to said charged molecule to form a complex; and
b) contacting said cell with said complex; wherein said charged molecule is taken up by said cell.
   In related embodiments, the present invention provides methods for delivering a charged molecule to a cell, said method comprising contacting said cell with a complex prepared by binding non-covalently a molecular entity comprising an amphiphilic core and at least two oppositely charged arms covalently attached thereto to said charged molecule, wherein said charged molecule is taken up by said cell.

In yet other embodiments, the present invention provides methods for stabilizing a charged molecule in vivo. The methods comprise contacting the charged molecule with a molecular entity comprising an amphiphilic core and at least two oppositely charged arms covalently attached thereto.

In yet other embodiments, the present invention provides methods for increasing the temperature of hybrid dissociation of a double-stranded or hairpin nucleic acid, said method comprising contacting said nucleic acid with a molecular entity comprising an amphiphilic core and at least two positively charged arms covalently attached thereto.

In yet other embodiments, the present invention provides methods for reducing the susceptibility of a double-stranded or hairpin nucleic acid to digestion by enzymatic nuclease, said method comprising contacting said nucleic acid with a molecular entity comprising an amphiphilic core and at least two positively charged arms covalently attached thereto. In preferred embodiments, the nuclease is exonuclease.

In yet other embodiments, the present invention provides methods for reducing the susceptibility of a double-stranded or hairpin nucleic acid to hydrolysis of the phosphodiester backbone, said method comprising contacting said nucleic acid with a molecular entity comprising an amphiphilic core and at least two positively charged arms covalently attached thereto.

In yet other embodiments, the present invention provides methods for reducing the susceptibility of charged molecules to self-aggregation, said method comprising contacting said charged molecule with a molecular entity comprising an amphiphilic core and at least two oppositely charged arms covalently attached thereto.

In some embodiments, the present invention provides compositions comprising a pharmaceutical excipient, a charged molecule and a molecular entity comprising an amphiphilic core and at least two oppositely charged arms covalently attached thereto, or a pharmaceutically acceptable ester, salt, or hydrate thereof.

As used herein, the term “pharmaceutical excipient” refers to an inert substance added to a pharmacological composition to further facilitate administration of molecular entities. Examples of pharmaceutical excipients include but are not limited to, calcium carbonate, calcium phosphate, various sugars and types of starch, cellulose derivatives, gelatin, vegetable oils and polyethylene glycols excipient.

As used herein, “pharmaceutically acceptable” refers to materials and compositions that are physiologically tolerable and do not typically produce an allergic or similar untoward reaction, such as gastric upset, dizziness, and the like, when administered to a human. Typically, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

As used herein, the term “pharmaceutical acceptable ester” within the context of the present invention represents an ester of a construct of the invention having a carboxy group, preferably a carboxylic acid prodrug ester that may be convertible under physiological conditions to the corresponding free carboxylic acid.

As used herein, the term “pharmaceutically acceptable salt” includes salts of acidic or basic groups that may be present in molecular entities used in the present compositions. Molecular entities included in the present compositions that are basic in nature are capable of forming a wide variety of salts with various inorganic and organic acids. The acids that may be used to prepare pharmaceutically acceptable acid addition salts of such basic molecular entities are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Molecular entities included in the present compositions that include an amino moiety may form pharmaceutically acceptable salts with various amino acids, in addition to the acids mentioned above. Molecular entities, included in the present compositions, which are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

The compositions according to the present invention may be administered to humans and other animals for therapy as either a single dose or in multiple doses. The compositions of the present invention may be administered either as individual therapeutic agents or in combination with other therapeutic agents. The treatments of the present invention may be combined with conventional therapies, which may be administered sequentially or simultaneously. In some embodiments, routes of administration include those selected from the group consisting of oral, intravesically, intravenous, intraarterial, intraperitoneal, local administration, and the like. Intravenous administration is the preferred mode of administration. It may be accomplished with the aid of an infusion pump.

The phrases “parenteral administration” and “administered parenterally” as used herein means modes of administration other than enteral and topical administration, usually by injection, and includes, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection, infusion, and the like.

The phrases “systemic administration,” “administered systemically,” “peripheral administration” and “administered peripherally” as used herein mean the administration of a compound, drug or other material by a route which does not introduce the compound, drug or other material directly into the central nervous system (for example, subcutaneous administration), such that it enters the patient's system and, thus, is subject to metabolism and other like processes.

Actual dosage levels of the active ingredients in the compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular molecular entities of the present invention employed, or the ester, salt or amide thereof, the route of administration, the time of administration, the rate of excretion of the particular compositions being employed, the duration of the treatment, other drugs, compounds and/or materials used in combination with the particular motor protein therapeutic employed, the age, sex, weight, condition, general health and prior medical history of the patient being treated, and like factors well known in the medical arts.

In general, a suitable daily dose of a compound of the invention will be that amount of the molecular entities which is the lowest dose effective to produce a therapeutic effect. Such an effective dose will generally depend upon the factors described above. Generally, intravenous, intracerebroventricular and subcutaneous doses of the compositions of the present invention for a patient will range from about 0.0001 to about 100 mg per kilogram of body weight per day.

In other embodiments, the present invention provides complexes comprising an anionic charged molecule associated with a molecular entity comprising an amphiphilic core and at least two positively charged arms covalently attached thereto. In preferred embodiments, the amphiphilic core is a biphenyl derivative. The ratio of the molecular entity to said anionic charged molecule ranges from about 1:1 to about 10:1; preferably ranges from about 1:1 to about 4:1.

In yet other embodiments, the present invention provides complexes comprising a cationic charged molecule associated with a molecular entity comprising an amphiphilic core and at least two negatively charged arms covalently attached thereto. In preferred embodiments, the amphiphilic core is a biphenyl derivative. The ratio of the molecular entity to said cationic charged molecule ranges from about 1:1 to about 10:1; preferably ranges from about 1:1 to about 4:1.

In yet other embodiments, the present invention provides compositions comprising a pharmaceutical excipient, an anionic charged molecule and a molecular entity comprising an amphiphilic core and at least two positively charged arms covalently attached thereto. The ratio of the entity to the anionic charged molecule in the composition may range from about 1:1 to about 10:1; preferably from about 1:1 to about 4:1. The anionic charged molecules may be a double-stranded or hairpin nucleic acid. The anionic charged molecules may be selected from the group consisting of single-stranded DNA, double-stranded DNA, single-stranded RNA, double-stranded RNA, oligonucleotide comprising non-natural monomers and the like. The single-stranded DNA, double-stranded DNA, single-stranded RNA and double-stranded RNA may include nucleotides bound to small molecules. In related embodiments, the single-stranded RNAs may be mRNA or miRNA and double-stranded RNA may be siRNA.

### Examples

The invention now being generally described, it will be more readily understood by reference to the following examples, which are included merely for purposes of illustration of certain aspects and embodiments of the present invention, and are not intended to limit the invention.

### Example 1

### Preparation of Oligopeptides

Oligopeptides such as oligolysine, oligoarginine or any suitable oligopeptide with amine moiety can be prepared via standard solid phase peptide synthesis. Examples used here may include any oligolysine up to twelve-mer.

### Example 2

### General Procedure for the Formation of Peptide Bond

To a solution of cyclodextrin compounds with free amino groups (1 eq) and C-terminus oligopeptide or simple amino acid with all amino groups protected as t-butyl carbamate (Boc) or 9-fluorenylmethyl carbamate (Fmoc) (2.2 eq) in anhydrous DMF in an ice bath was added hydroxybenzotriazole (HOBt) (2.2 eq). The resulting solution was stirred at 0° C. for 30 min. Dicyclohexylcarbodiimide (DCC) (2.2 eq) was then added. The mixture was stirred at 0° C. to room temperature until the reaction was complete (monitored by HPLC). The precipitated dicyclohexylurea (DCU) was filtered off and the filtrate was concentrated under reduced pressure. The residue was slurried with ethyl acetate and then filtered or decanted. The solid containing the desired compound and DCU was used in the next step without further purification.

### Example 3

### General Procedure for the Deprotection of Boc Protected Amino Group

The Boc protected amino compound was dissolved in trifluoroacetic acid (TFA) and dichloromethane (25%). The resulting solution was stirred at room temperature for 0.5-3 hours. The solvent was evaporated under reduced pressure and the residue was dissolved in water. The undissolved DCU was filtered off and the filtrate was evaporated under reduced pressure to give the desired compound.

### Example 4

### General Procedure for the Deprotection of Fmoc Protected Amino Group

The Fmoc protected amino compound was dissolved in DMF and the piperidine was added. The resulting solution was stirred at room temperature for several hours until the protecting group was completely removed (monitored by HPLC). The solvent was evaporated under reduced pressure and the residue was dissolved in water, filtered and washed with ethylacetate. The aqueous phase was evaporated to dryness to give the desired product.

### Example 5

### A Linear Core System with Oligopeptide Arm

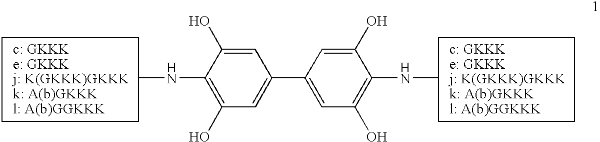

The peptide can be prepared according to the procedures in the art, see e.g. Examples 1-4. Reaction of 4,4′-diaminobiphenyl-3,3′,5,5′-tetraol with desired protected peptide under suitable amide bond formation condition affords molecular entities 1 with two oligopeptide arms that comprise amine side chains capable of forming positive charge.

### Example 6

### Procedure A: Couple with Peptide

To a solution of a linear core (1 eq) and C-terminus oligopeptide building block or simple amino acid with all amino group protected by t-Butyl carbamate (Boc) or 9-Fluorenylmethyl Carbamate (Fmoc) (2.2 eq) in anhydrous DMF at room temperature was added coupling agents (DIC or TBTU or HATU and HObt) (2.2 eq) and diisopropylamine (DIPEA) (2.2 eq). The resulting solution was stirred at ambient temperature until completion (monitored by HPLC). The solution was concentrated under reduced pressure. The residue was washed with water and ethyl acetate. The compound was further purified by preparative HPLC if necessary. Refer to the general procedure in Example 2 if DCC was used as the coupling agent.

### Example 7

### Procedure B: Deprotection of Fmoc Protected Amino Group

The Fmoc protected amino compound was dissolved in 20% piperidine/DMF. The resulting solution was stirred at room temperature for 0.5-1 hour until the protecting group was completely removed (monitored by HPLC). The solvent was removed under reduced pressure and the residue was mixed with water to form a slurry. The resulting slurry was filtered, and the filtrate was washed with ethyl acetate and dried to give the desired product. The product was used to the next step without further purification.

### Example 8

### Procedure C: Deprotection of Boc Protected Amino Group

The Boc protected amino compound was dissolved in methylene chloride-trifluoroacetic acid solution (3:1). The resulting solution was stirred at rt for 0.5-1 hour. The solvent was then evaporated under reduced pressure to give a TFA salt. If necessary, the TFA salt can be converted to a HCl salt by dissolving the compound in 1 M HCl solution and then evaporated to dryness two times. The overall yields from coupling to the final product were from 5% to 90%. The products were further purified by preparative HPLC, if needed.

### Example 9

### Procedure D: Couple with Alkyl Carboxylic Acid (or Activated NHS Ester)

The same procedure in Example 10 was used to couple with alkylcarboxylic acids or NHS activated esters in the presence of DIPEA (2.2 eq) in DMF.

### Example 10

### Procedure E: Couple with Cross Linking Reagent

A compound with free amino groups (1 eq) was dissolved in DMF, after the cross linking reagent (NHS-R-MAL) (2.5 eq) and DIPEA (2.5 eq) were added to the reaction solution, the resulting reaction mixture was stirred at room temperature until completion of the reaction (monitored by HPLC). The reaction solution was concentrated under reduced pressure and the residue was washed with water and ethyl acetate. The crude product was used without further purification.

### Example 11

### Procedure F: General Procedure for the Reaction Between Maleimide Group and Thiol Group

A compound with maleimide group (1 eq) was dissolved in a mixed solvent of methanol-1 M Tris buffer (pH 7.2) (ratio 4:1). The solution was degassed and the peptide with a free thiol group (2.5 eq) was added to the solution. After the reaction was complete (monitored by HPLC), the solvent was removed and the residue was purified by preparative HPLC to give product.

### Example 12

### Preparation of 2

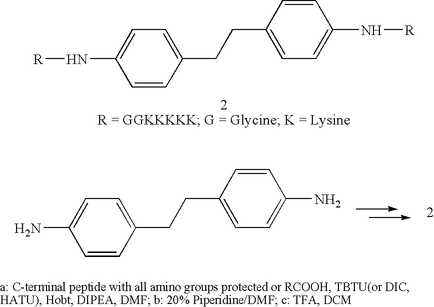

Compound 2 was synthesized following the general procedures for each step as follows: coupled 4,4′-(ethane-1,2-diyl)dianiline with Fmoc-Gly-Gly-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); Boc deprotection (procedure C). Compound 2 was isolated as the HCl salt. MS (ESP) m/z calcd for C₈₂H₁₄₈N₂₆O₁₄ 1722, Found 1723.

### Example 13

### Preparation of 3-6

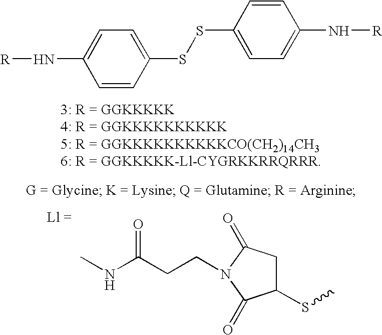

### Example 13-1

### Preparation of Compound 3

Compound 3 was synthesized following the general procedures as described for each step as follows: coupled 4,4′-disulfanediyldianiline with Fmoc-Gly-Gly-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection procedure B); Boc deprotection (procedure C). The compound 3 was isolated as the HCl salt. ¹HNMR (300 MHz, D₂O): δ 1.20-2.00 (m, 60H), 2.80-2.95 (m, 20H), 3.9-4.25 (m, 8H), 4.5 (br, 10H), 7.30 (d, 4H), 7.45 (d, 4H); MS (MALDI) m/z calcd for C₈₂H₁₄₈N₂₆O₁₄S₂ 1752, Found 1763 (M+Na)⁺.

### Example 13-2

### Preparation of Compound 4

Compound 4 was synthesized following the general procedures as described for each step as follows: coupled 4,4′-disulfanediyldianiline with Fmoc-Gly-Gly-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 4 was isolated as the HCl salt. ¹HNMR (300 MHz, D₂O): δ 1.20-2.00 (m, 120H), 2.80-2.95 (m, 40H), 3.9-4.25 (m, 8H), 4.5 (br, 20H), 7.30 (d, 4H), 7.45 (d, 4H); MS (MALDI) m/z calcd for C₁₄₀H₂₆₄N₄₆O₂₄S₂ 3038, Found 3040.

### Example 13-3

### Preparation of Compound 5

Compounds 5 was synthesized following the general procedures as described for each step as follows: coupled 4,4′-disulfanediyldianiline with Fmoc-Gly-Gly-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); further coupled with CH₃(CH₂)₁₄COOH (procedure A); Boc deprotection (procedure C). The compound 5 was isolated as the HCl salt. ¹HNMR (300 MHz, D₂O): δ 0.75 (t, 6H), 1.10-1.85 (m, 154H), 2.20 (t, 4H), 2.80-2.95 (m, 40H), 3.85-4.00 (m, 8H), 4.10-4.25 (m, 20H), 7.30 (d, 4H), 7.45 (d, 4H); MS (MALDI) m/z calcd for C₁₇₂H₃₂₄N₄₆O₂₆S₂ 3038, Found 3040.

### Example 13-4

### Preparation of Compound 6

Compounds 6 was synthesized following the general procedures as described for each step as follows: coupled 4,4′-disulfanediyldianiline with Fmoc-Gly-Gly-OH (procedure A): Fmoc deprotection (procedure B); further coupled with Fmoc-Lys(Boc)-Lys(Boc)-Lys(Boc)-Lys(Boc)-Lys(Boc)-OH (procedure A), Fmoc deprotection (procedure B); coupled with NHS-3-maleimideopropionate (procedure D); coupled with CYGRKKRRQRRR (CTAT) (procedure E); Boc deprotection (procedure C). The compound 6 was isolated as the HCl salt. ¹HNMR (300 MHz, D₂O): δ 1.00-2.00 (m, 100H), 2.10-2.60 (m, 12H), 2.80-2.95 (m, 32H), 3.05-3.15 (m, 24H), 3.5-4.00 (m, 18H), 4.25 (br, 22H), 6.75 (d, 4H), 7.05 (d, 4H), 7.30 (d, 4H), 7.45 (d, 4H), 7.5 (d, 4H), 7.7 (d, 4H).

### Example 14

### Selective 6-OH Protection of a Beta-CD and Corresponding AD-Ring Homologation

The primary hydroxyl groups at the A,D-rings of β-CD can be readily protected by reaction of β-CD with biphenyl-4,4′-disulfonyl dichloride in the presence of amine base such as pyridine according to known procedures (Tabushi et al., J. Am. Chem. Soc. 1984, 106, 5267-5270). The desired compound 7 may be purified by suitable means, e.g. by reverse phase column chromatography. Amine moiety can be readily introduced at 6 position of A,D-rings. Compound 7 reacts with NaN₃ in DMF followed by triphenylphosphine (Ph₃P) reduction of azido groups to give desired compound 9.

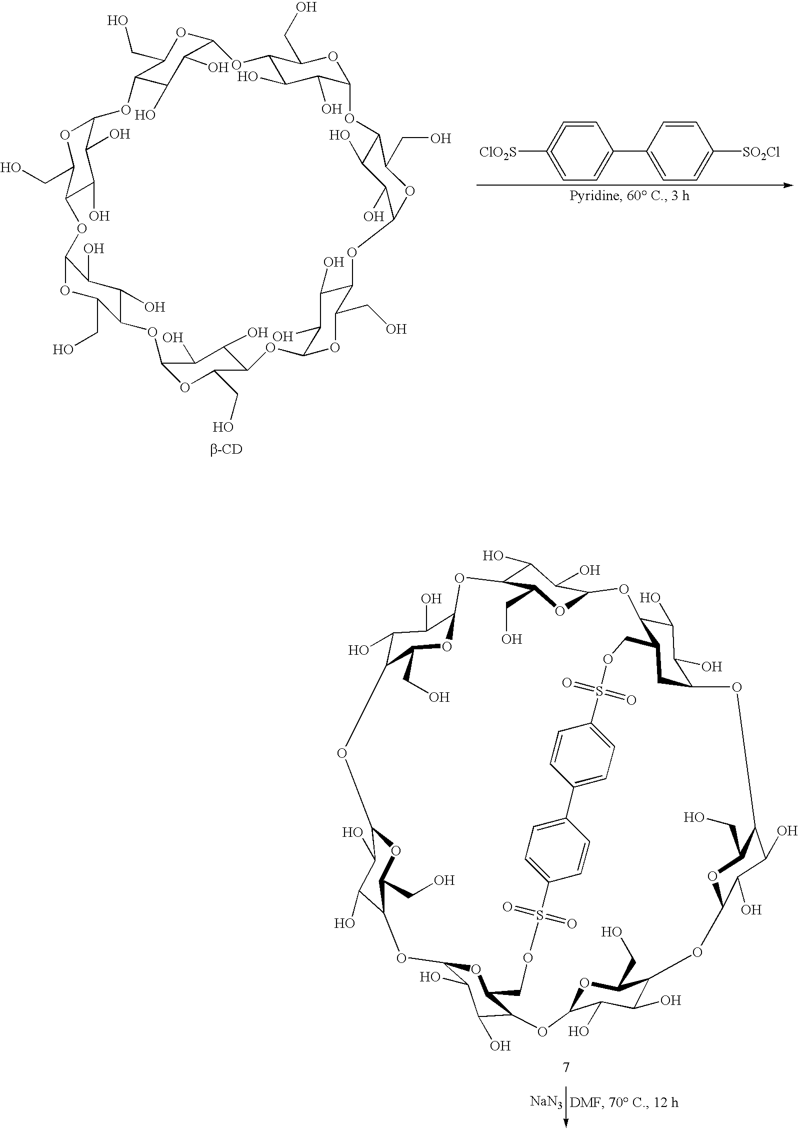

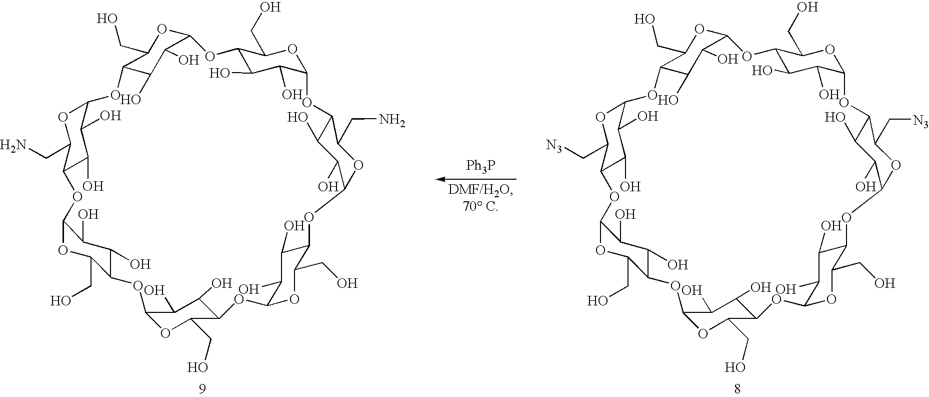

Alternatively, the procedure disclosed by Sinay et al. (Angew. Chem. Int. Ed. Engl., 2000, 39, 3610-3612) can be employed to selectively establish the 6^{A},6^{D}-ring functionality. Per-benzyl β-CD 10 is reduced at the 6^{A},6^{D}-ring to give 11 and subsequently to diamine 14 via mesylation (compound 12), azido conversion (compound 13) and azido reduction

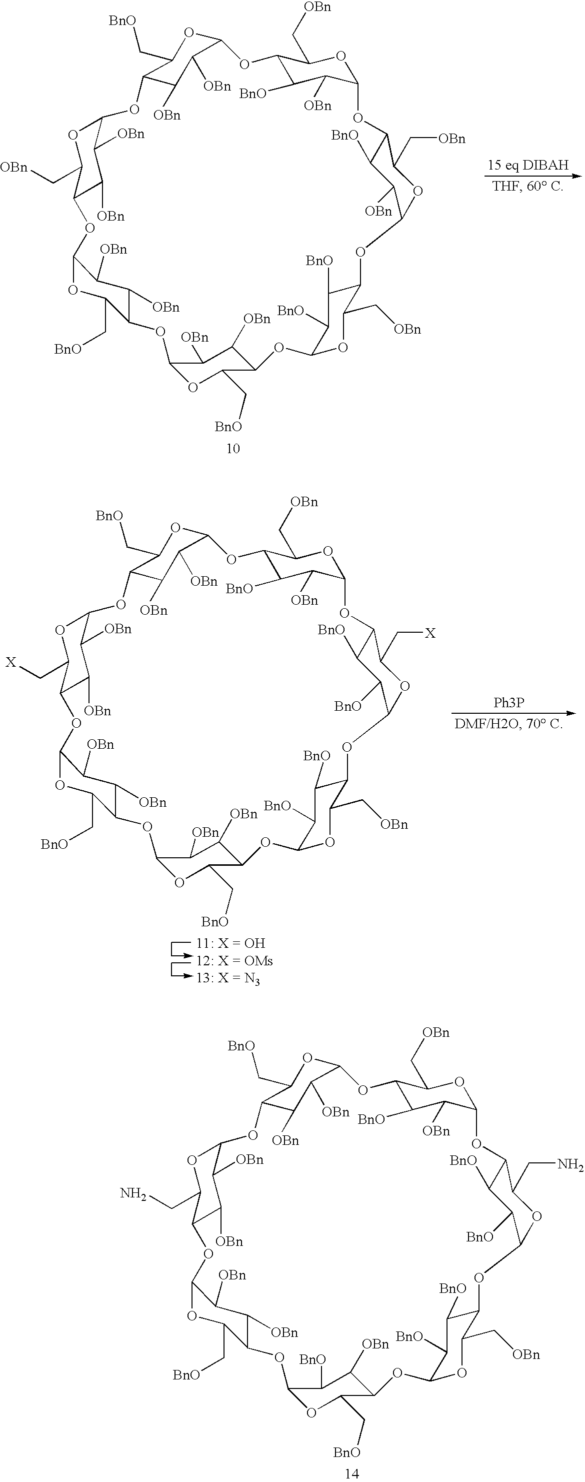

### Example 15

### Introduction of Substituents at 6^{A},6^{D} Ring Protected β-CD

Introduction of substituents at the 6-positions of A,D rings of β-CD can be achieved by discriminating the reactivity of the 2,3 positions versus the 6 position. All the 6 hydroxyl groups of β-CD are protected selectively with t-butyldimethytlsilylchloride (TBDMSCl) to give 15 followed by exhaustive benzylation of the remaining positions and deprotection of the 6-position resulting in 16. Alkylation of 16 with PMB-chloride affords 17 displaying two sets of orthogonal protecting groups. 17 is selectively reduced to 18 followed by two step functional group conversion to 20. Selective deprotection of 20 with acid gives 21 which can be selectively derivatized at the 6-position of B,C,E,F, and G rings by a skilled artisan to afford 22. Finally, reduction with trimethylphosphine (Me₃P) results in 23.

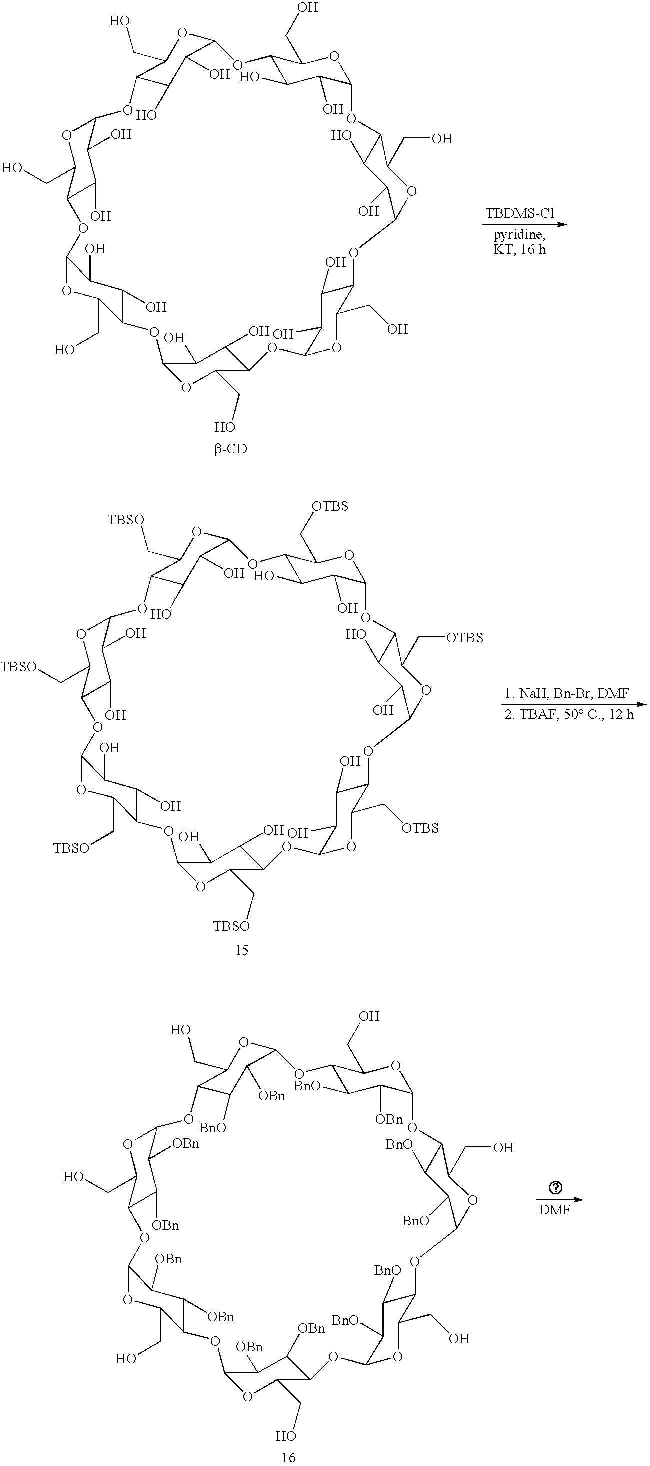

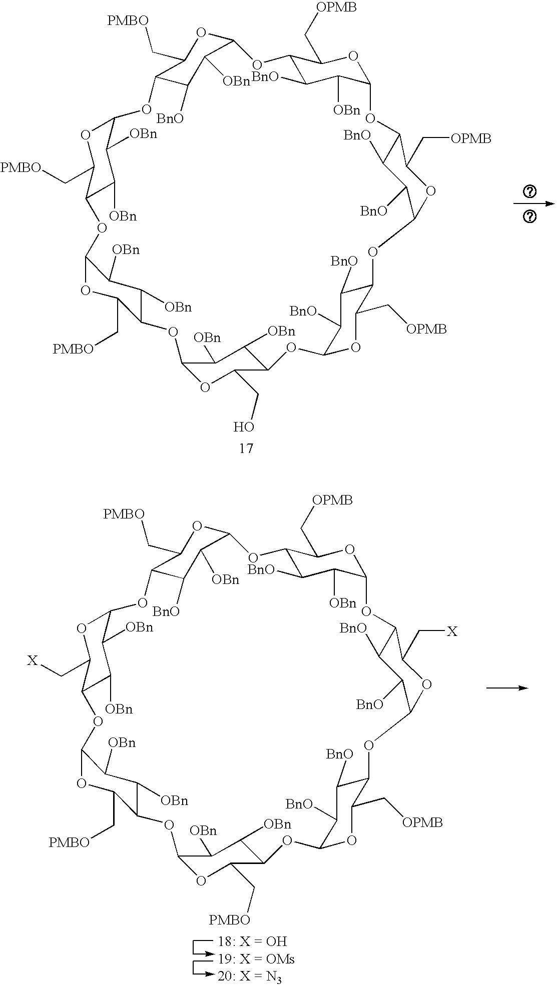

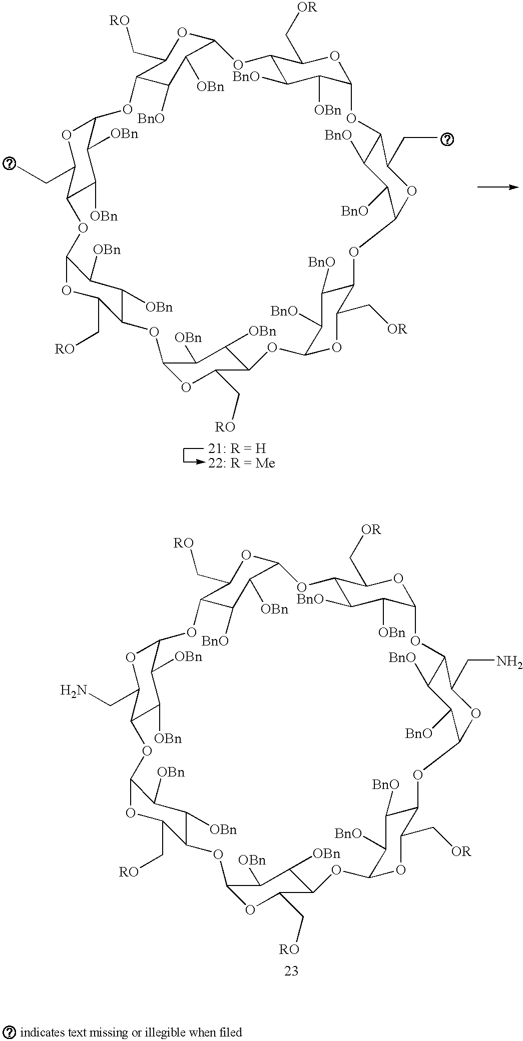

### Example 16-1

### Preparation of Compound 17

To a suspension of NaH (2.31 g, 57.83 mmols) in DMF (30 mL) at 0° C. under nitrogen was added a solution of 16 (9.90 g, 4.13 mmols) in DMF (50 mL) via syringe. The mixture was stirred at 0° C. for 10 minutes and at room temperature for 10 minutes. The mixture was re-cooled to 0° C. and PMBCl (7.85 mL, 57.83 mmols) was added drop wise via syringe. Stirring was continued and the mixture was warmed to room temperature overnight. The reaction mixture was cooled to 0° C., quenched with water slowly and concentrated under vacuum. The residue was dissolved in ethyl acetate and the organic phase was washed with 0.1 N aqueous HCl, followed by saturated aqueous NaHCO₃ and brine. The organic phase was then dried over anhydrous MgSO₄, filtered and concentrated under vacuum. The residue was purified by flash chromatography on silica gel employing hexanes and ethyl acetate as the eluting solvents to give 11.200 g (83%) of 17. ¹H NMR (300 MHz, CDCl₃): δ 3.41-3.50 (m, 14H), 3.60 (s, 21H), 3.7-5.2 (m, 77H), 6.70-7.41 (m, 98H).

### Example 16-2

### Preparation of Compound 18

The product 11 (6.70 g, 2.07 mmols) from above and molecular sieves (9 g, 4 Å) were transferred into a flame-dried flask and kept under nitrogen. Dry toluene was added via syringe and the mixture equilibrated at 40° C. for 10 minutes. DIBAH (69 mL, 103.46 mmols) in toluene was added via syringe and the reaction was stirred for 45 minutes. The reaction mixture was cooled to −10° C. in an acetone/ice bath and carefully quenched with water. Ethyl acetate was added to the resulting suspension and then filtered through celite. The precipitate was further washed with hot ethyl acetate and the filtrates were combined. The combined filtrate was washed with brine, dried over anhydrous MgSO₄, filtered and concentrated under vacuum. The residue was purified by flash chromatography on silica gel employing hexanes and ethyl acetate as the eluting solvents to give 3.20 g (52%) of 18 as a white solid. ¹H NMR (300 MHz, CDCl₃): δ 3.41-5.60 (m, 104H), 6.70-7.70 (m, 90H).

### Example 16-3

### Preparation of Compound 19

A solution of 18 (2.00 g, 0.67 mmols) in dry pyridine (30 mL) under nitrogen was cooled to 0° C. and MsCl (0.26 mL, 3.34 mmols) was added via syringe. The reaction mixture was stirred to room temperature overnight and concentrated under vacuum at room temperature. The residue was taken up in ethyl acetate and washed with 0.1 N aqueous HCl, saturated aqueous NaHCO₃, brine, dried over anhydrous MgSO₄, filtered and concentrated under vacuum. The residue was purified by flash chromatography on silica gel employing hexanes and ethyl acetate as the eluting solvents to give 1.90 g (90%) of 18. ¹H NMR (300 MHz, CDCl₃): δ 2.60 (s, 6H), 3.20-3.50 (m, 8H), 3.65 (s, 15H), 3.65-5.40 (m, 79H), 6.60-7.60 (m, 90H).

### Example 16-4

### Preparation of Compound 20

NaN₃ (0.59 g, 9.03 mmols) was added to a solution of 19 (1.90 g, 0.60 mmols) in DMF (25 mL). The reaction mixture was stirred at 80° C. for 20 h, concentrated under vacuum, and treated with ethyl acetate. The ethyl acetate solution was washed with water, brine, dried over anhydrous MgSO₄, filtered and concentrated under vacuum to give 1.75 g (95%) of 20. ¹H NMR (300 MHz, CDCl₃): δ 3.30-3.70 (m, 20H) 3.70 (s, 15H), 3.75-4.2 (bs, 24H), 4.30-4.60 (m, 25H), 4.70 (bs, 9H), 4.90-5.40 (m, 9H), 6.70-7.70 (m, 90H).

### Example 16-5

### Preparation of Compound 21

10% TFA in dichloromethane (27 mL) was added to compound 20 (1.50 g, 0.49 mmols) at room temperature. The mixture was stirred at room temperature for 20 minutes and slowly added to saturated aqueous NaHCO₃ solution. The organic layer was separated and the aqueous phase extracted with dichloromethane (5 mL×5). The combined organic extracts were dried over anhydrous MgSO₄, filtered and concentrated under vacuum. The residue was purified by flash chromatography on silica gel employing 5% methanol in ethyl acetate as the eluting solvent to give 0.50 g (42%) of 21. ¹H NMR (300 MHz, CDCl₃): δ 2.90-4.25 (m, 47H), 4.30-5.50 (m, 35H), 7.20 (bs, 70H).

### Example 16-6

### Preparation of Compound 22

To a suspension of NaH (0.08 g, 2.04 mmols) in DMF (2 mL) at 0° C. and under nitrogen was added a mixture of 21 (0.40 g, 0.16 mmols) and MeI (0.13 mL, 2.04 mmols) in DMF (8 mL) via syringe. The mixture was stirred at 0° C. for 1 h and at room temperature for another 1 h. The mixture was re-cooled to 0° C., quenched with methanol and concentrated under vacuum. The residue was dissolved in dichloromethane, washed with water, aqueous Na₂S₂O₃, brine, dried over anhydrous MgSO₄, filtered and concentrated under vacuum. The residue was purified by flash chromatography on silica gel employing hexanes and ethyl acetate as the eluting solvents to give 0.244 g (59%) of 22. ¹H NMR (300 MHz, CDCl₃): δ 3.35 (s, 15H), 3.40-4.10 (m, 42H), 4.30-4.70 (m, 12H), 4.70-5.30 (m, 38H), 7.20 (bs, 70H).

### Example 16-7

### Preparation of Compound 23

To a solution of 22 (0.23 g, 0.09 mmols) in THF/0.1N NaOH; 9:1 (10 mL) at room temperature was added Me₃P (0.82 mL, 0.82 mmols). The resulting reaction mixture was stirred overnight and then concentrated under vacuum. The residue was taken up in ethyl acetate and washed with saturated aqueous NaHCO₃, brine, dried over anhydrous MgSO₄, filtered and concentrated under vacuum. The residue was purified by flash chromatography on silica gel employing 10% methanol in dichloromethane as the eluting solvent to give 0.080 g (36%) of 23. ¹H NMR (300 MHz, CDCl₃): δ 2.90-3.20 (bs, 4H), 3.35 (s, 15H), 3.35-3.60 (m, 13H), 3.70-4.15 (m, 27H), 4.30-5.40 (m, 37H), 7.20 (bs, 70H).

### Example 17

### Introduction of Substituents at 2,3 Positions of A,D Ring of β-CD

Selective silylation of the primary hydroxyl groups of 8 gives 24 followed by exhaustive derivatization of the 2,3-positions using excess reagent as shown below for the methylation of 24 to arrive at 25. Desilylation of 25 and subsequent reduction of 26 with Ph₃P gives diamine 27 ready for final assembly with cationic arms.

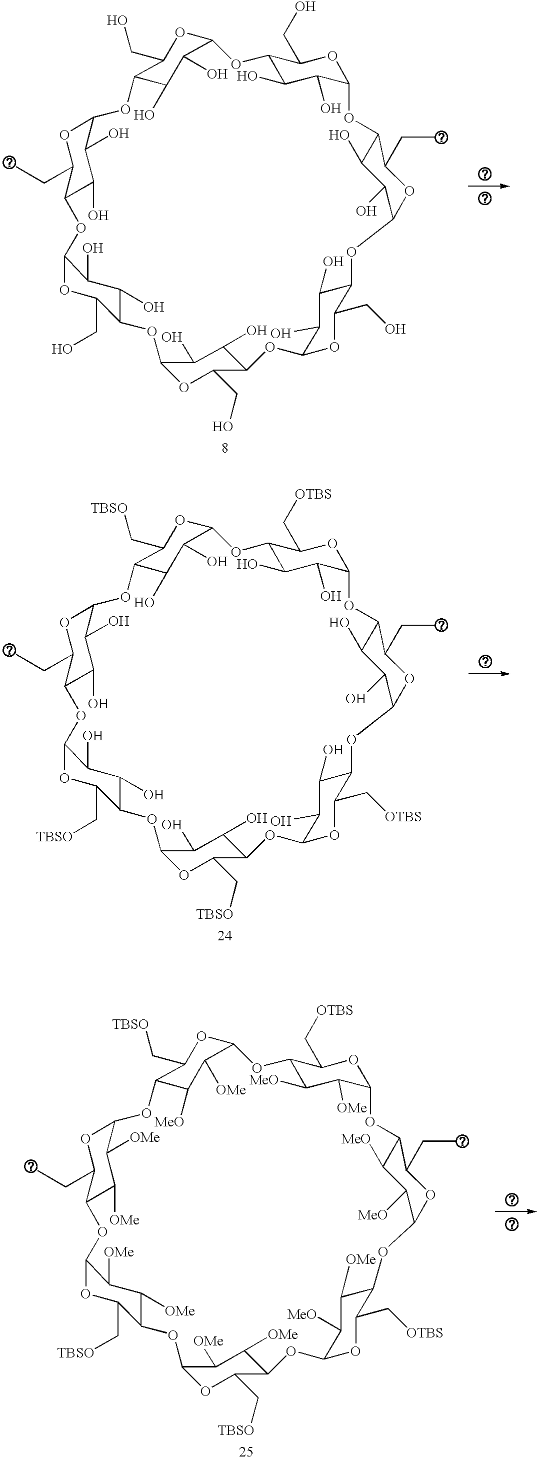

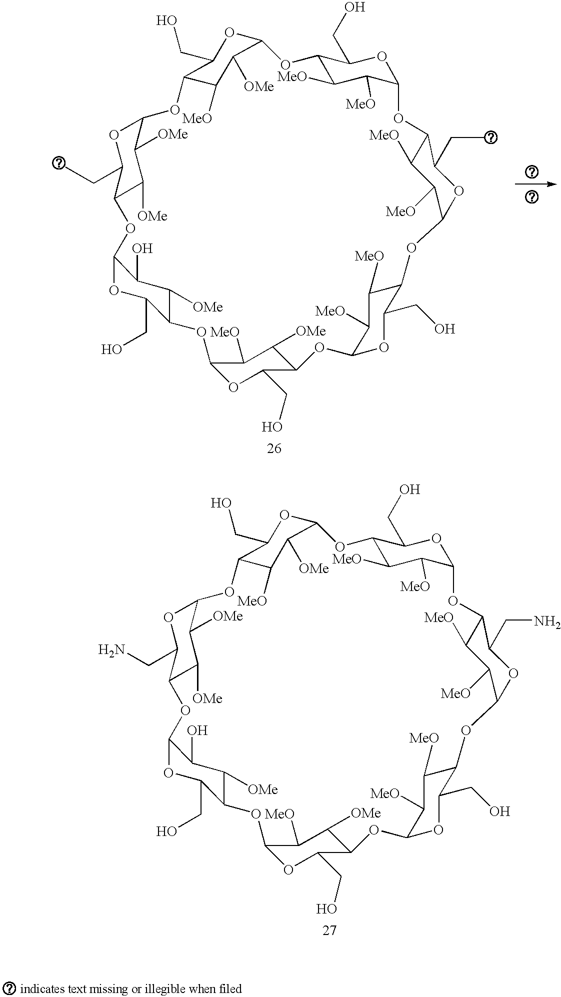

### Example 17-1

### Preparation of Compound 24

To solution of 95 mg (0.080 mmol) of 8 in 1 ml absolute pyridine was added 84 mg (0.56 mmol) t-BDMSCl. The reaction mixture was stirred for 18 h at room temperature and then concentrated at vacuum. The semi crystalline residue was taken up in a few drops of methanol, re-precipitated from an excess of water and finally washed with ethyl acetate. Upon drying in vacuo 125 mg (89%) colorless precipitate was obtained. ¹H NMR (300 MHz, CDCl₃): δ −0.1-0.0 (30H), δ 0.95-1.10 (45H), 3.25-4.05 (m, 42H), 4.8-4.95 (m, 7H).

### Example 17-2

### Preparation of Compound 25

To a suspension of NaH (100 mg, 2.5 mmols) in DMF (3 mL) at 0° C. under nitrogen was added a solution of 24 (120 mg, 0.068 mmols) in DMF (2 mL) via syringe. The mixture was stirred at 0° C. for 10 minutes and at room temperature for 10 minutes. The mixture was re-cooled to 0° C. and methyliodide (0.125 ml, 2.0 mmols) was added drop wise via syringe. Stirring was continued and the mixture was warmed to room temperature overnight. After cooling the reaction mixture to 0° C. it was slowly quenched with water and concentrated under vacuum. The residue was dissolved in ethyl acetate and the organic phase was washed with 0.1 N aqueous HCl, followed by saturated aqueous NaHCO₃ and brine. Drying over anhydrous MgSO₄, followed by filtration and concentration under vacuum gave an oily residue which was purified by flash chromatography on silica gel employing hexanes and ethyl acetate as the eluting solvents to give 75 mg (57%) of 25. ¹H NMR (300 MHz, CDCl₃): δ 0.0 (s, 30H), δ 0.82 (s, 45H), 2.95-3.18 (m, 7H), 3.3-4.2 (m, 84H) 5.02-5.25 (m, 7H).

### Example 17-3

### Preparation of Compound 26

HBF₄ was added via syringe to compound 25 (0.42 g, 0.21 mmols) in acetonitrile (13 mL) solution in a polyethylene container at room temperature. The mixture was stirred for 1 h at room temperature, quenched with saturated aqueous NaHCO₃ solution and extracted several times with dichloromethane. The extracts were combined, washed with brine, dried over anhydrous MgSO₄, filtered and concentrated under vacuum to give 0.230 g (77%) of 26. ¹H NMR (300 MHz, CDCl₃): δ 3.20 (bs, 9H), 3.30-4.00 (78H), 5.1 (m, 9H).

### Example 17-4

### Preparation of Compound 27

To 26 (0.20 g, 0.15 mmols) dissolved in DMF (5 mL) and H₂O (0.5 mL) was added prewashed solid supported Ph₃P (0.29 g, 0.88 mmols; 3 mmols/g loading). The mixture was stirred at 60° C. overnight, the resin filtered-off, and the filtrate was concentrated under vacuum. The residue was dissolved again in DMF (5 mL) and H₂O (0.5 mL) and 10 eq. of supported Ph₃P (0.48 g, 1.46 mmols; 3 mmols/g loading) added. The mixture was heated at 70° C. overnight, filtered off resin and the filtrate concentrated under vacuum. The residue was purified by flash chromatography on silica gel employing 2% NH₄OH/20% methanol in dichloromethane as the eluting solvent to give 0.100 g (51%) of 27. ¹H NMR (300 MHz, CDCl₃): δ 3.00-4.00 (91H), 5.1 (m, 9H).

### Example 18

### Preparation of 6^{A},6^{D} Ring β-CD with Mercapto Linker

The 6^{A},6^{D} di-iodo β-CD can be prepared according to known procedures (Hwang et al., Bioconjugate Chem. 2001, 12, 280). Compound 28 can be prepared by reaction of 7 with KI in DMF at 80° C. for 2 hours. Compound 28 is then readily available for derivatization via nucleophilic substitution to give thioether 29.

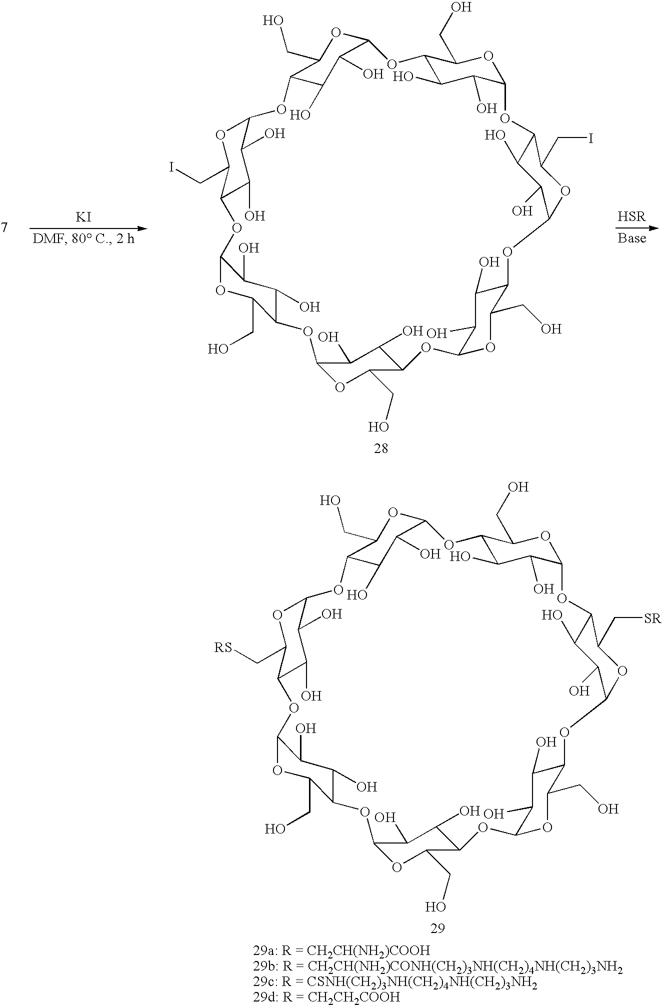

### Example 18-1

### Preparation of Compound 29a

KOH (0.1 g, 1.5 mmol, 10 eq) was added to a solution of compound 28 (0.2 g, 0.15 mmol) in DMF (2 ml). After being purged with nitrogen, Boc-Cys (89 mg, 0.44 mmol, 3.3 eq) was added to the reaction mixture and then purged again with nitrogen. The resulting reaction mixture was stirred at room temperature for 24 h. The solvent was removed under reduced pressure and the residue was washed with water, ethyl acetate and then was dried under vacuum to yield product 23a as a white solid (0.22, 80%). ¹H-NMR (300 MHz, D₂O) δ 1.25-1.5 (s, 18H), 3.2-4.1 (br, 48H), 4.85-5.00 (s, 7H).

### Example 18-2

### Preparation of Compound 29b

Compound 29b was synthesized employing similar procedures for the formation of amide bond and the subsequent deprotection of Boc group using compound 29a (0.1 g, 0.065 mmol) and NH₂(CH₂)₃N(Boc)(CH₂)₄N(Boc)(CH₂)₃NH(Boc) (0.068 g, 0.135 mmol, 2 eq) to yield product 29b (70 mg, 63%). ¹H-NMR (300 MHz, D₂O) δ 1.00-2.0 (m, 16H), 2.8-4.0 (m, 72H), 5.00 (s, 7H).

### Example 18-3

### Preparation of Compound 29c

To a solution of compound 28 (0.1 g, 0.075 mmol) and NH₂(CH₂)₃N(Boc)(CH₂)₄N(Boc)(CH₂)₃NH(Boc) (90 mg, 0.18 mmol, 2.4 eq) were added K₃PO₄ (165 mg, 0.72 mmol, 4.8 eq) and carbon disulfide (43 μl, 0.72 mmol, 4.8 eq). The resulting mixture was stirred at ambient temperature for 24 h. The solvent was evaporated and the residue was dissolved in water and then washed with ethyl acetate. The aqueous solution was evaporated to dryness and then slurried with water to provide a solid compound after drying under reduced pressure. The dried compound was dissolved in 75% TFA/CH₂Cl₂ and stirred for 3 h. The solvent was evaporated under reduced pressure to yield product 29c as a pale yellow solid (80 mg, 46%). ¹H-NMR (300 MHz, D₂O) δ 1.00-2.0 (m, 16H), 3.0-4.2 (m, 66H), 5.00 (s, 7H).

### Example 18-4

### Preparation of Compound 29d

To a solution of 28 (0.200 g, 0.147 mmols) in DMF (4 mL) was added 3-mercaptopropionic acid (0.128 mL, 1.476 mmols) and NEt₃ (0.103 mL, 0.738 mmols) at room temperature and under nitrogen. The mixture was heated at 60° C. overnight with stirring. The mixture was concentrated to near dryness and acetone added. The precipitate formed was further washed with acetone, 5% water in acetone and dried under vacuum at 60° C. for 5 h to give 29d (0.165 g, 85%) as an off-white solid. ¹H NMR (300 MHz, DMSO-d₆): δ 2.55-3.10 (m, 7H), 3.50-4.10 (bs, 35H), 4.10-4.70 (m, 6), 4.70-5.20 (m, 10H), 5.40-6.30 (m, 18H).

### Example 19

### Preparation of 6^{A},6^{D} Ring-Derivatized CD with Di-Thioether Linker

Reaction of 6^{A},6^{D}-diamino β-CD with dithioether containing compounds will lead to β-CD substituted with di-thioether linkers. For example, starting with compound 9, dithiodiglycolic acid will give compounds with dithioether bridges such as 30. Compound 30 can then be selectively coupled to the amino terminus of an oligopeptide. A skilled artisan also can prepare other derivatives following procedures known in the art.

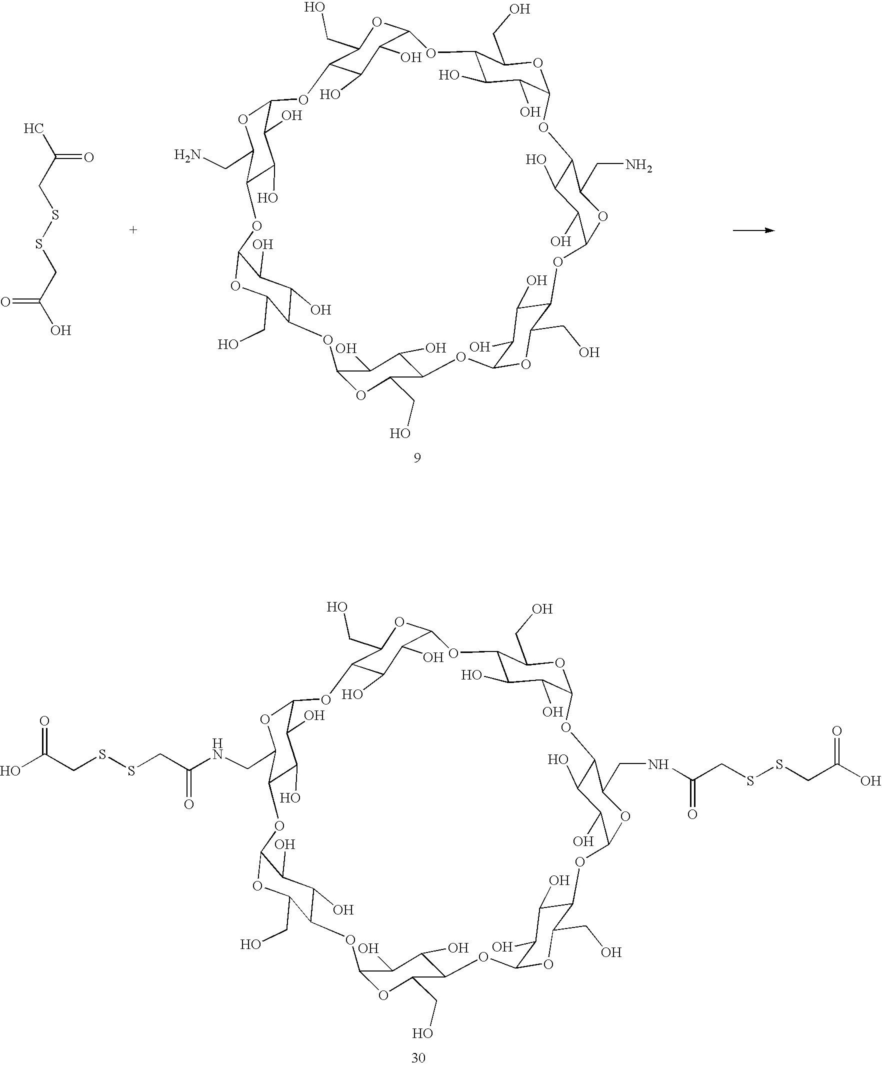

### Example 20

### Synthesis of Oligopeptide-Cyclodextrin Conjugates 31

Reaction of compounds 9, 14, 23, and 27 with the C-terminus of an oligopeptide affords compounds 31. Upon removal of protecting groups such as Boc or Cbz, the desired construct suitable to complex with siRNA can be readily prepared.

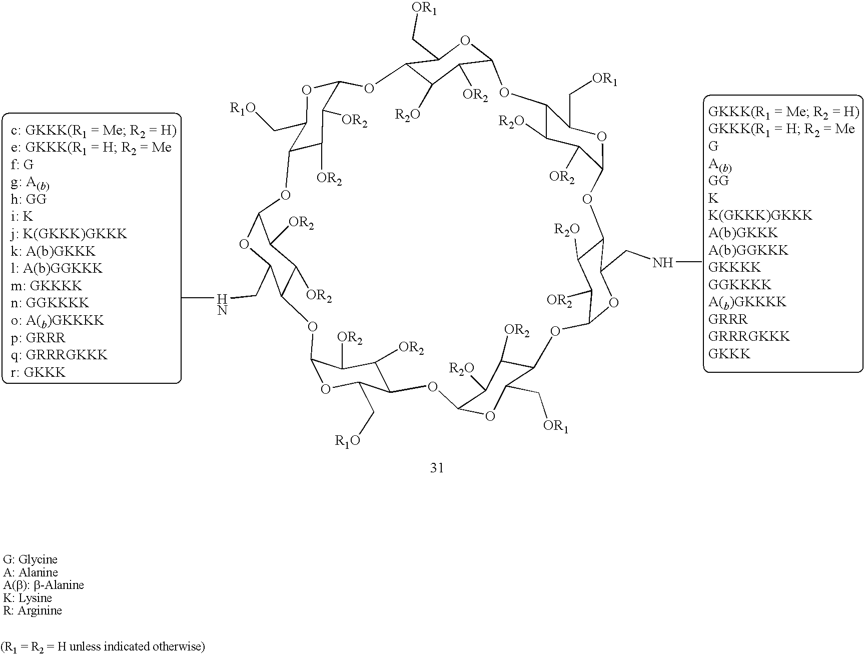

### Example 20-1

### Preparation of Tetramer Peptide CD Conjugate 31a

To a solution of 23 (0.08 g, 0.03 mmols) and Boc-Lys(Boc)-Lys(Boc)-Lys(Boc)-Gly-OH (0.07 g, 0.08 mmols) in DMF was added HOBt (0.01 g, 0.08 mmols) and DCC (0.02 g, 0.08 mmols) at room temperature. The mixture was stirred at room temperature overnight and an additional DCC (10 mg) and HOBt (8 mg) was added. The reaction was further stirred at room temperature overnight, concentrated to near dryness under vacuum and the residue was treated with ethyl acetate. The organic phase was washed with saturated aqueous NaHCO₃, brine, dried over anhydrous MgSO₄, filtered and concentrated under vacuum. The residue was purified by flash chromatography on silica gel employing 10% methanol in dichloromethane as the eluting solvent to give 0.112 g (36%) of 31a. ¹H NMR (300 MHz, CDCl₃): δ 1.40 (s, 72H), 1.55-6.10 (m, 218H), 7.20 (m, 90H).

### Example 20-2

### Preparation of Tetramer Peptide CD Conjugate 31b

To the above compound 31a (0.11 g, 0.03 mmols) in THF (10 mL) was added 10% Pd/C and palladium black (0.03 g). The reaction mixture was evacuated and flushed three times with a hydrogen filled balloon before stirring was continued for 48 h. The reaction mixture was filtered through celite and the catalyst (10% Pd/C and palladium black) was washed with THF. The filtrate was concentrated, treated with acetone and the precipitate washed several times with acetone. The precipitate was then dried under vacuum at 60° C. overnight to give 0.063 g (84%) of 31b. MS m/z Calcd for (M+H)⁺ C₁₂₇H₂₂₄N₁₆O₅₇: 2887.21; Found: 2888.00.

### Example 20-3

### Preparation of Tetramer Peptide CD Conjugate 31c

To compound 31b (0.04 g, 0.015 mmols) was added 75% TFA in dichloromethane (3 mL) and the resulting reaction mixture was stirred at room temperature for 2.5 h. The mixture was concentrated under vacuum, triturated with cyclohexane and the precipitate collected by filtration. The precipitate was then dried under vacuum at 60° C. overnight to give 0.047 g 100%) of 31c. MS m/z Calcd for (M+H)⁺ C₈₇H₁₆₀N₁₆O₄₁: 2086.28; Found: 2087.40.

### Example 20-4

### Preparation of Tetramer Peptide CD Conjugate 31d

To a solution of 27 (0.05 g, 0.04 mmols) and Boc-Lys(Boc)-Lys(Boc)-Lys(Boc)-Gly-OH (0.08 g, 0.09 mmols) in DMF was added HOBt (0.01 g, 0.09 mmols) and DCC (0.02 g, 0.08 mmols) at room temperature. The mixture was stirred at room temperature overnight under nitrogen, concentrated to near dryness under vacuum and the residue treated with ethyl acetate. The organic phase was washed with saturated aqueous NaHCO₃, brine, dried over anhydrous MgSO₄, filtered and concentrated under vacuum. The residue was purified by flash chromatography on silica gel employing 10% methanol in dichloromethane as the eluting solvent to give 0.025 g (22%) of 31d. ¹H NMR (300 MHz, CDCl₃): δ 0.60-0.90 (m, 5H), 1.10-1.50 (bs, 117H), 1.50-1.70 (bs, 8H), 1.97 (s, 5H), 2.20 (bs, 9H), 2.90-3.25 (m, 24H), 3.30-3.65 (m, 59H), 3.65-4.00 (m, 4H), 4.80-5.20 (m, 11H).

### Example 20-5

### Preparation of Tetramer Peptide CD Conjugate 31e

To compound 31d (0.02 g, 0.001 mmols) was added 75% TFA in dichloromethane (5 mL) and the resulting reaction mixture was stirred at room temperature for 1.5 h. The mixture was concentrated under vacuum, triturated with cyclohexane and the precipitate collected by filtration. The precipitate was then dried under vacuum at 50° C. for 48 h to give 0.025 g 100%) of 31e. MS m/z Calcd for (M+H)⁺ C₉₆H₁₇₈N₁₆O₄₁: 2212.52; Found: 2213.50.

### Example 20-6

### Preparation of 6^{A},6^{D}-dideoxy-6^{A},6^{D}-di(Gly)amino-β-cyclodextrin (31f)

Compound 31f was synthesized as described in the general procedures for the formation of CD-peptide bond and the subsequent deprotection of Fmoc group using 6^{A},6^{D}-dideoxy-6^{A},6^{D}-diamino-β-cyclodextrin (9) (0.4 g, 0.35 mmol) and Fmoc-glycine (0.228 g, 0.77 mmol, 2.2 eq) to yield product 31f (0.35 g, 80%) as a pale yellow solid. ¹H-NMR (300 MHz, D₂O) δ 3.0-4.0 (m, 46H), 5.08 (s, 7H).

### Example 20-7

### Preparation of 6^{A},6^{D}-dideoxy-6^{A},6^{D}-di(β-Ala)amino-β-cyclodextrin (31g)

Compound 31g was synthesized as described in the general procedures for the formation of CD-peptide bond and the subsequent deprotection of Fmoc group using compound 9 (0.4 g, 0.35 mmol) and Fmoc-β-alanine (0.24 g, 0.77 mmol, 2.2 eq) to yield product 31g (0.12 g, 27%) as a off-white solid. ¹H-NMR (300 MHz, DMSO-d₆) δ 3.0-4.3 (m, 75H), 4.80-4.90 (m, 7H).

### Example 20-8

### Preparation of 6^{A},6^{D}-dideoxy-6^{A},6^{D}-di(Gly-Gly)amino-β-cyclodextrin (31h)

Compound 31h was synthesized as described in the general procedures for the formation of CD-peptide bond and the subsequent deprotection of Fmoc group using compound 31f (0.5 g, 0.39 mmol) and Fmoc-glycine (0.260 g, 0.87 mmol, 2.2 eq) to yield product 31h (0.2 g, 37%) as pale yellow solid. ¹H-NMR (300 MHz, D₂O) δ 3.0-4.0 (m, 50H), 4.99 (s, 7H); MS m/z Calcd. for C₅₀H₈₄N₆O₃₇ 1360.49, Found 1361.7.

### Example 20-9

### Preparation of 6^{A},6^{D}-dideoxy-6^{A},6^{D}-di(Lys)amino-β-cyclodextrin (31i)

Compound 31i was synthesized as described in the general procedures for the formation of the CD-peptide and the subsequent deprotection of Boc group using compounds 9 (0.1 g, 0.085 mmol) and Boc-Lys(Boc)-OH (0.077 g, 0.185 mmol, 2.2 eq) to yield product 31i (0.04 g, 34%) as a pale yellow solid. ¹H-NMR (300 MHz, D₂O) δ 1.48-1.65 (m, 12H), 2.87 (t, 4H), 3.26-3.95 (m, 44H), 4.95 (s, 7H).

### Example 20-10

### Preparation of 6^{A},6^{D}-dideoxy-6^{A},6^{D}-di[Lys-(Gly-Lys-Lys-Lys-NH₂)-Gly-Lys-Lys-Lys-NH₂]amino-β-cyclodextrin (31j)

Compound 31j was synthesized as described in the general procedures for the formation of the CD-peptide and the subsequent deprotection of Boc group using compound 31i (0.020 g, 0.014 mmol), Boc-Lys(Boc)-Lys(Boc)-Lys(Boc)-Gly-OH (0.054 g, 0.063 mmol, 4.5 eq) and compound 9 to yield 50 mg product 31j (50 mg, 71%) as a pale yellow oil. ¹H-NMR (300 MHz, CD₃OD) δ 1.05-2.00 (m, 84H), 2.75-3.00 (m, 28H), 3.26-3.953, 30-4.40 (m, 64H), 4.95 (s, 7H, merged with H₂O peak).

### Example 20-11

### Preparation of 6^{A},6^{D}-dideoxy-6^{A},6^{D}-di(Ala(β)-Gly-Lys-Lys-Lys-NH₂)amino-β-cyclodextrin (31k)

Compound 31k was synthesized as described in the general procedures for the formation of CD-peptide and the subsequent deprotection of Boc group using compound 31g (0.020 g, 0.0156 mmol) and Boc-Lys(Boc)-Lys(Boc)-Lys(Boc)-Gly-OH (0.030 g, 0.0348 mmol, 2.2 eq) to yield product 31k (40 mg, 81%) as a pale yellow solid. ¹H-NMR (300 MHz, D₂O) δ 1.05-2.00 (m, 36H), 2.30-4.2 (m, 72H), 4.95 (s, 7H); MS m/z Calcd for C₈₈H₁₆₀N₁₈O₄₃ 2158.3, Found 1080.41 [M+2]⁺⁺/2.

### Example 20-12

### Preparation of 6^{A},6^{D}-dideoxy-6^{A},6^{D}-di(Ala(β)-Gly-Gly-Lys-Lys-Lys-NH₂)amino-β-cyclodextrin (31l)

Compound 31l was synthesized as described in the general procedures for the formation of CD-peptide bond and the subsequent deprotection of Boc group using compound 31g (0.020 g, 0.0156 mmol) and Boc-Lys(Boc)-Lys(Boc)-Lys(Boc)-Gly-Gly-OH (0.030 g, 0.0348 mmol, 2.2 eq) to yield product 31l (17 mg, 53%) as an off white solid. ¹H-NMR (300 MHz, D₂O) δ 1.05-2.00 (m, 36H), 2.30-4.2 (m, 76H), 4.95 (s, 7H); MS m/z Calcd for C₉₂H₁₆₆N₂₀O₄₅ 2272.4, Found 1137.23 [M+2]⁺⁺/2.

### Example 20-13

### Preparation of 6^{A},6^{D}-dideoxy-6^{A},6^{D}-di(Gly-Lys-Lys-Lys-Lys-NH₂)amino-β-cyclodextrin (31m)

Compound 31m was synthesized as described in the general procedures for the formation of CD-peptide and the subsequent deprotection of Boc group using compound 9 (0.020 g, 0.0175 mmol) and Boc-Lys(Boc)-Lys(Boc)-Lys(Boc)-Lys(Boc)-Gly-OH (0.042 g, 0.0386 mmol, 2.2 eq) to yield product 31m (26 mg, 43%) as a white solid. ¹H-NMR (300 MHz, D₂O) δ 1.25-2.00 (m, 36H), 2.70-4.2 (m, 68H), 4.95 (s, 7H); MS m/z Calcd for C₉₂H₁₆₆N₂₀O₄₅ 2158.3, Found 1137.23 [M+2]⁺⁺/2.

### Example 20-14

### Preparation of 6^{A},6^{D}-dideoxy-6^{A},6^{D}-di(Gly-Gly-Lys-Lys-Lys-Lys-NH₂)amino-β-cyclodextrin (31n)

Compound 31n was synthesized as described in the general procedures for the formation of CD-peptide and the subsequent deprotection of Boc group using compound 31f (0.040 g, 0.032 mmol) and Boc-Lys(Boc)-Lys(Boc)-Lys(Boc)-Lys(Boc)-Gly-OH (0.076 g, 0.070 mmol, 2.2 eq) to yield product 31n (15 mg, 13%) as a off white solid. ¹H-NMR (300 MHz, D₂O) δ 1.25-2.00 (m, 48H), 2.80-4.2 (m, 74H), 4.95 (s, 7H).

### Example 20-15

### Preparation of 6^{A},6^{D}-dideoxy-6^{A},6^{D}-di(Ala(β)-Gly-Lys-Lys-Lys-Lys-NH₂)amino-β-cyclodextrin (31o)

Compound 31o was synthesized as described in the general procedures for the formation of CD-peptide bond and the subsequent deprotection of Boc group using compound 31g (0.020 g, 0.016 mmol) and Boc-Lys(Boc)-Lys(Boc)-Lys(Boc)-Lys(Boc)-Gly-OH (0.038 g, 0.035 mmol, 2.2 eq) to yield product 31o (14 mg, 25%) as a off white solid. ¹H-NMR (300 MHz, D₂O) δ 1.25-2.00 (m, 48H), 2.30-4.2 (m, 78H), 4.95 (s, 7H); MS m/z Calcd for C₁₀₀H₁₈₄N₂₂O₄₅ 2414.65, Found 1208.33 [M+2]⁺⁺/2.

### Example 20-16

### Preparation 6^{A},6^{D}-dideoxy-6^{A},6^{D}-di(Gly-Arg-Arg-Arg-NH₂)amino-β-cyclodextrin (31p)

Compound 31p was synthesized as described in the general procedures for the formation of CD-peptide bond and the subsequent deprotection of Fmoc group using compound 9 (0.030 g, 0.026 mmol) and Fmoc-Arg-Arg-Arg-Gly-OH (0.046 g, 0.06 mmol, 2.2 eq) to yield product 31p (50 mg, 88%) as an oil. ¹H-NMR (300 MHz, D₂O) δ 1.40-2.00 (m, 24H), 3.00-4.25 (m, 64H), 4.95 (s, 7H); MS m/z Calcd for C₈₂H₁₅₀N₂₈O₄₁ 2184.23, Found 1092.45 [M+2]⁺⁺/2.

### Example 20-17

### Preparation 6^{A},6^{D}-dideoxy-6^{A},6^{D}-di(Gly-Arg-Arg-Arg-Gly-Lys-Lys-Lys-NH₂)amino-β-cyclodextrin (31q)

Compound 31q was synthesized as described in the general procedures for the formation of CD-peptide bond and the subsequent deprotection of Boc group using compound 31p (0.043 g, 0.02 mmol) and Boc-Lys(Boc)-Lys(Boc)-Lys(Boc)-Gly-OH (0.037 g, 0.044 mmol, 2.2 eq) to yield product 31q (20 mg, 21%) as a pale yellow solid. ¹H-NMR (300 MHz, D₂O) δ 1.15-2.00 (m, 60H), 3.00-4.25 (m, 86H), 4.95 (s, 7H); MS m/z Calcd for C₁₂₂H₂₂₈N₄₂O₄₉ 3067.37, Found 1023.28[M−3]⁺⁺⁺/3.

### Example 20-18

### Preparation 6^{A},6^{D}-dideoxy-6^{A},6^{D}-di[Gly-Lys(Boc)-Lys(Boc)-Lys(Boc)-Boc]amino-nonadecakis-O-benzyl-β-cyclodextrin (31r)

To a solution of 6^{A},6^{D}-dideoxy-6^{A},6^{D}-diamino-nonadecakis-O-benzyl-β-cyclodextrin (14) (0.1 g, 0.035 mmol) in anhydrous DMF (5 mL) were added HOBt (10.8 mg, 0.08 mmol), compound Boc-Lys(Boc)-Lys(Boc)-Lys(Boc)-Gly-OH (0.072 g, 0.084 mmol, 2.4 eq) and DCC (0.017 g, 0.084 mmol, 2.4 eq). The resulting solution was stirred at ambient temperature for 24 hours. The solvent was evaporated to dryness and the residue was dissolved in water/ethyl acetate and filtered. The organic phase was washed with water and brine. The solution was dried (MgSO₄), filtered and evaporated. The residue was purified by column chromatography on silica gel column using dichloromethane as an eluent to provide product 31r (100 mg, 63%). ¹H-NMR (300 MHz, CDCl₃) δ 1.10-2.00 (m, 108H), 2.85-5.25 (m, 123H), 6.90-7.40 (m, 95H).

### Example 20-19

### Preparation 6^{A},6^{D}-dideoxy-6^{A},6^{D}-di[Gly-Lys(Boc)-Lys(Boc)-Lys(Boc)-Boc]amino-β-cyclodextrin (31s)

To a solution of compound 31r (0.3 g, 0.066 mmol) in 11 mL of mixed solvent of ethanol and acetic acid (10.1) was added 10% Pd/C (350 mg). The suspension was purged with nitrogen and stirred under hydrogen (balloon) at room temperature for one day. The reaction mixture was filtered through a cellite pad and washed with methanol and water. The filtrate was evaporated and the residue was washed with cyclohexane. The product was dried under vacuum to provide product 31s (110 mg, 66%). ¹H-NMR (300 MHz, CD₃OD) δ 1.10-2.00 (m, 108H), 2.85-4.25 (m, 64H), 4.95 (s, 7H).

### Example 20-20

### Preparation 6^{A},6^{D}-dideoxy-6^{A},6^{D}-di(Gly-Lys-Lys-Lys-NH₂)amino-β-cyclodextrin (31t)

A solution of compound 31s (0.1 g, 0.036 mmol) in a mixed solvent of trifluoroacetic acid (TFA, 3 mL) and dichloromethane (1 mL) was stirred at ambient temperature for 3 hours. The solvent was evaporated to provide a quantitative yield of product 31t as a TFA salt. ¹H-NMR (300 MHz, D₂O) δ 1.10-2.00 (m, 36H), 2.85-4.25 (m, 64H), 4.95 (s, 7H). MS m/z Calcd for C₈₂H₁₅₀N₁₆O₄₁ 2016.15, Found 1008.67 [M+2]⁺⁺/2.

### Example 21

### Synthesis of Oligoamine-Cyclodextrin Conjugates 31u to 31z

Similar to the synthesis of oligopeptide-cyclodextrin conjugates, oligoamines were used as the cationic arms to prepare oligoamine-cyclodextrin conjugates. Reaction of compound 9 with the unprotected amine of an oligoamine afforded compounds 31u to 31z. Upon removal of protecting groups such as Boc or Cbz, the desired constructs suitable to complex with siRNA can be readily prepared.

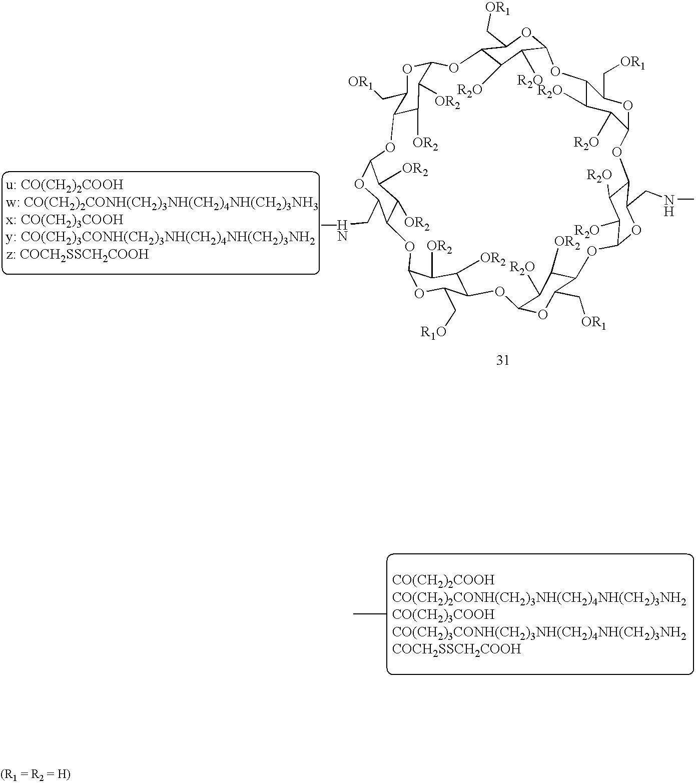

### Example 21-1

### Preparation of Compound 31u

To a solution of 9 (0.500 g, 0.440 mmols) in DMF (8 mL) was added succinic anhydride (0.093 g, 0.933 mmols) at room temperature and under nitrogen. Stirring was continued for 1 h, concentrated to ˜3 mL volume and acetone was added. The precipitate formed was further washed with acetone and dried under vacuum at 50° C. overnight to give 31u (0.570 g, 97%) as an off-white solid. ¹H NMR (300 MHz, D₂O): δ 2.30-2.65 (m, 11H), 3.05-3.40 (m, 5H), 3.40-3.65 (m, 18H), 3.65-3.95 (m, 47H), 4.95-5.10 (s, 7H).

### Example 21-2

### Spermine Coupling to Succinamide-Cyclodextrin—Preparation of Compound 31v

To a solution of 31u (0.160 g, 0.120 mmols) and H₂N(CH₂)₃NHBoc(CH₂)₄NHBoc(CH₂)₃NHBoc (0.145 g, 0.288 mmols) in DMF (6 mL) under nitrogen was added HOBt (0.039 g, 0.288 mmols) and DCC (0.059 g, 0.288 mmols) at room temperature and stirred for 4 h. Thereafter, HOBt (0.039 g, 0.288 mmols) and DCC (0.059 g, 0.288 mmols) were added and the reaction stirred at room temperature overnight, concentrated to near dryness under vacuum and the residue treated with dichloromethane. The precipitate obtained was further washed with dichloromethane several times and dried under vacuum at room temperature overnight to give 31v (0.138 g, 50%) as an off-white solid). ¹H NMR (300 MHz, DMSO-d₆): δ 1.30-1.50 (s, 54H), 1.50-1.8 (m, 12H), 2.15-2.45 (m, 9H), 2.80-3.25 (m, 25H), 3.50-3.80 (bs, 24H), 4.35-4.52 (bs, 5H), 4.52-5.00 (bs, 9H), 5.55-6.10 (bs, 15H), 6.60-6.80 (bs, 3H), 7.55-7.85 (m, 4H).

### Example 21-3

### Preparation of Compound 31w

To the above compound 31v (0.124 g, 0.054 mmols) was added 75% TFA in dichloromethane (5 mL) and stirred at room temperature for 3 h. The mixture was concentrated under vacuum, treated with water and extracted with dichloromethane (5 mL×2). The aqueous solution was lyophilized to give 0.070 g 76%) of 31w as an off-white solid. ¹H NMR (300 MHz, DMSO-d₆): δ 1.40-1.80 (bs, 11H), 1.90 (s, 6H), 2.10-2.45 (m, 9H), 2.65-3.20 (m, 26H), 3.50-4.00 (bs, 28H), 4.50-4.70 (bs, 6H), 4.85 (s, 9H), 5.40-6.15 (bs, 15H), 7.70 (s, 2H), 7.80-8.30 (m, 8H), 8.45-9.10 (m, 8H).

### Example 21-4

### Preparation of Compound 31x

To a solution of 9 (0.500 g, 0.440 mmols) in DMF (3 mL) was added glutaric anhydride (0.127 g, 1.113 mmols) at room temperature and under nitrogen. Stirring was continued for 2.5 h, concentrated to near dryness and added ethyl acetate. The precipitate formed was further washed with ethyl acetate and dried under vacuum at 60° C. for 2 h to give 31x (0.574 g, 96%) as an off-white solid. ¹H NMR (300 MHz, DMSO-d₆): δ 1.50-1.90 (m, 6H), 2.00-2.30 (m, 10H), 3.50-3.95 (bs, 30H), 4.20-4.70 (m, 6H), 4.85 (s, 9H), 5.30-6.20 (bs, 18H), 7.40-7.80 (m, 3H).

### Example 21-5

### Preparation of Compound 31y

Compound 31y was synthesized as described in the procedure for the coupling of spermine to derivatized cyclodextrin (see above for Step A and B) and the subsequent removal of the Boc group using compound 31x (0.200 g, 0.147 mmols), H₂N(CH₂)₃NHBoc(CH₂)₄NHBoc(CH₂)₃NHBoc (0.177 g, 0.353 mmols), HOBt (0.059 g, 0.441 mmols) and DCC (0.091 g, 0.441 mmols) to give 31y (0.124 g, 88%) as an off-white solid. ¹H NMR (300 MHz, DMSO-d₆): δ 1.30-1.80 (m, 15H), 1.90 (s, 3H), 2.10 (s, 7H), 2.60-3.20 (bs, 22H), 3.40 (s, 15H), 3.80-4.60 (b, 26H), 4.85 (s, 9H), 5.30-6.20 (b, 14H), 7.45-7.80 (m, 3H), 7.97 (s, 9H), 8.40-9.10 (m, 9H).

### Example 21-6

### Preparation of Compound 31z

To a solution of 9 (0.400 g, 0.353 mmols) and dithiodiglycolic acid (0.322 g, 1.760 mmols) in DMF (10 mL) under nitrogen was added HOBt (0.114 g, 0.847 mmols) and DCC (0.175 g, 0.847 mmols) at room temperature and stirred for 5 h, concentrated to near dryness under vacuum and the residue treated with absolute ethanol. The precipitate obtained was sonicated, filtered and further washed with absolute ethanol several times and dried under vacuum at 55° C. overnight. The crude product was purified on reverse HPLC (Phenomenex Luna 5u, C18(2) column) to give 31z (0.064 g, 12%) as an off-white solid. MS m/z Calcd for C₅₀H₈₀N₂O₃₉S₄ 1461.42, Found 1461.98.

### Example 22

### Preparation of Compound 33-36

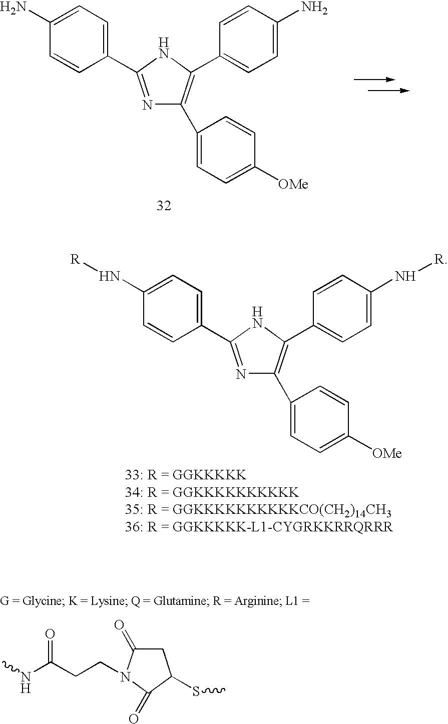

### Example 22-1

### Preparation of Compound 33

Compound 33 may be synthesized following the general procedures as described for each step as follows: couple compound 32 (prepared by reduction of the nitro precursors as described in Ultrasonics Sonochemistry, 2008, 15(5), 659-664) with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 33 is isolated as the HCl salt.

### Example 22-2

### Preparation of Compound 34

Compound 34 may be synthesized following the general procedures as described for each step as follows: couple compound 32 with Fmoc-Gly-Gly-OH procedure A); Fmoc deprotection procedure B); further couple with Fmoc-Lys(Boc)-Lys(Boc)-Lys(Boc)-Lys(Boc)-Lys(Boc)-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 34 is isolated as the HCl salt.

### Example 22-3

### Preparation of Compound 35

Compounds 35 may be synthesized following the general procedures as described for each step as follows: couple compound 32 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); further couple with CH₃(CH₂)₁₄COOH (procedure A); Boc deprotection procedure C). The compound 35 is isolated as the HCl salt.

### Example 22-4

### Preparation of Compound 36

Compounds 36 may be synthesized following the general procedures as described for each step as follows: couple compound 32 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); couple with NHS-3-maleimideopropionate (procedure D); couple with CYGRKKRRQRRR (CTAT) (procedure E); Boc deprotection (procedure C). The compound 36 is isolated as the HCl salt.

### Example 23

### Preparation of Compound 38-41

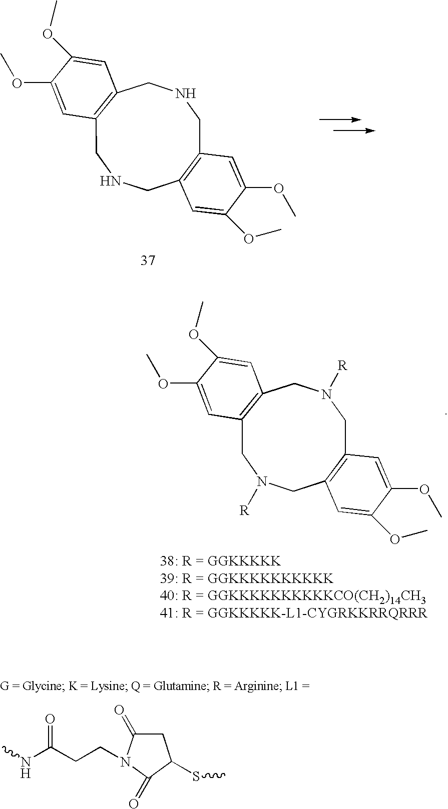

### Example 23-1

### Preparation of Compound 38

Compound 38 may be synthesized following the general procedures as described for each step as follows: couple compound 37 (known compound as described in Daiichi Coll. Pharm. Sci., Fukuoka, Japan. Heterocycles, 1987, 26(9), 2385-91) with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection; c. Boc deprotection (procedure B). The compound 33 is isolated as the HCl salt.

### Example 23-2

### Preparation of Compound 39

Compound 39 may be synthesized following the general procedures as described for each step as follows: couple compound 37 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 34 is isolated as the HCl salt.

### Example 23-3

### Preparation of Compound 40

Compounds 40 may be synthesized following the general procedures as described for each step as follows: couple compound 37 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); further couple with CH₃(CH₂)₁₄COOH (procedure A); Boc deprotection (procedure C). The compound 35 is isolated as the HCl salt.

### Example 23-4

### Preparation of Compound 41

Compounds 41 may be synthesized following the general procedures as described for each step as follows: couple compound 37 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); couple with NHS-3-maleimideopropionate (procedure D); couple with CYGRKKRRQRRR (CTAT) procedure E); Boc deprotection (procedure C). The compound 6 is isolated as the HCl salt.

### Example 24

### Preparation of Compounds 43-46

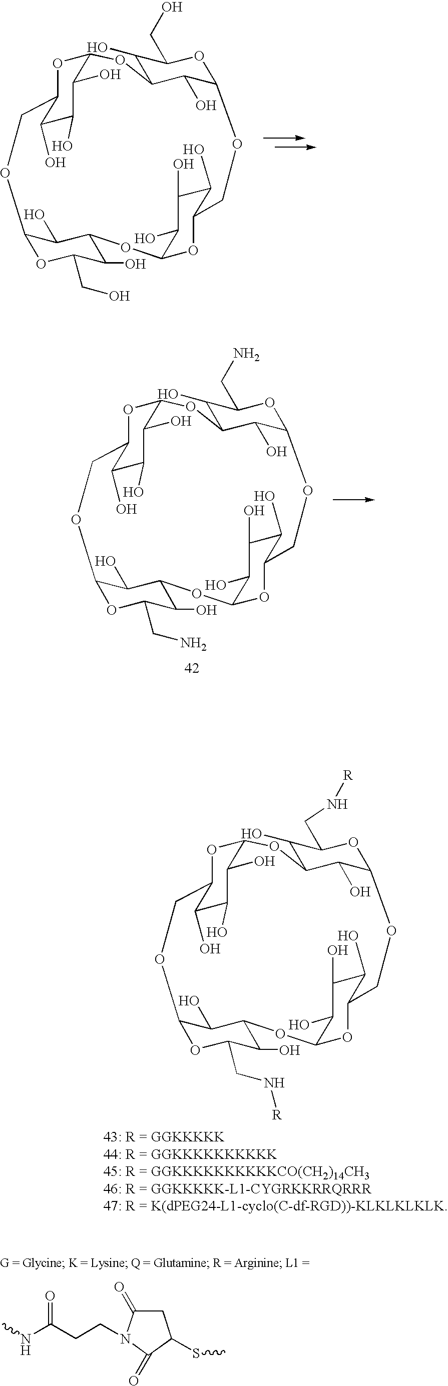

### Example 42-1

### Preparation of Compound 43

Compound 43 may be synthesized following the general procedures as described for each step as follows: couple compound 42 (which may be prepared from the known cyclo{-6)-α-D-Glcp-(1,3)-α-D-Glcp-(1,6)-α-D-Glcp-(1,3)-α-D-Glcp-1-} via 6-hydroxyl conversion to 6-amine) with Fmoc-Gly-Gly-OH procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 33 is isolated as the HCl salt.

### Example 24-2

### Preparation of Compound 44

Compound 44 may be synthesized following the general procedures as described for each step as follows: couple compound 42 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 34 is isolated as the HCl salt.

### Example 24-3

### Preparation of Compound 45

Compounds 45 may be synthesized following the general procedures as described for each step as follows: couple compound 42 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); further couple with CH₃(CH₂)₁₄COOH procedure A); Boc deprotection (procedure C). The compound 35 is isolated as the HCl salt.

### Example 24-4

### Preparation of Compound 46

Compounds 46 may be synthesized following the general procedures as described for each step as follows: couple compound 42 with Fmoc-Gly-Gly-OH (procedure A); Fmoc deprotection (procedure B); further couple with Fmoc-Lys(Boc)-Lys(Boc)-Lys(Boc)-Lys(Boc)-Lys(Boc)-OH (procedure A); Fmoc deprotection (procedure B); couple with NHS-3-maleimideopropionate (procedure D); couple with CYGRKKRRQRRR (CTAT) (procedure E); Boc deprotection (procedure C). The compound 6 is isolated as the HCl salt.

### Example 24-5

### Preparation of Compound 47

Compounds 47 may be synthesized following the general procedures as described for each step as follows: couple compound 42 with Fmoc-Lys(Boc)-Leu-Lys(Boc)-Leu-Lys(Boc)-Leu-Lys(Boc)-Leu-Lys(Boc)-OH (procedure A). To a solution of the resulting product in DMF, Pd(PPh₃)₄ (0.1 eq) and Me₂NH BH₃ complex (2.2 eq) are added. The reaction mixture is purged with nitrogen and stirred under nitrogen for 2 days until all Alloc and Fmoc groups are removed (monitored by HPLC). The solvent is removed and the residue is suspended in water. The aqueous suspension is washed with ether (3×) and lyophilized to give crude amino compound. To a solution of the crude amino compound in DMF, DIEA (10 eq) and NHS-dPEG₂₄-MAL (Quanta) (6 eq) are added and the reaction mixture is stirred for 12 h. The reaction mixture is diluted with phosphate buffer (50 mM NaHPO₄, 10 mM EDTA, and pH 7.2) in MeOH and cyclo(C-dF-RGD) peptide (7 eq) is added. The reaction mixture is purged with nitrogen and stirred for 2 days under nitrogen. The solvent is evaporated under reduced pressure and the residue is washed with water (2×) and then subject to the general procedure C to remove Boc groups. After removal of the solvent, the residue is purified by HPLC to give compound 47.

### Example 25

### siRNA Binding Assay

The relative binding affinity for each TCPC compound was monitored by both gel mobility shift and dye exclusion (see Morgan, A. R., Evans, D. H., Lee J. S., and Pulleyblank, D. E. 1979. Review: Nucl. Acids Res. 1979, 7, 571-594.) assays. Gel mobility shift assays were performed essentially as described as follows (see Parker, G. S., Eckert, D. M., and Bass, B. L. RNA. 2006, 12, 807-818.): Samples of ten or twenty-microliter scale with 50 pM end ³²P-labeled siRNA and various TCPC concentrations were incubated for 15 min at room temperature in a buffer containing a final concentration of 20 mM Tris pH 8.0, 150 mM NaCl, and 10% glycerol. Gel shifts assays of these samples were applied on 10% native gels electrophoresed at 4° C. RNA complexes were visualized using a Molecular Dynamics Typhoon PhosphorImager and apparent affinities were calculated as previously described. (see Parker, G. S., Eckert, D. M., and Bass, B. L. RNA. 2006, 12, 807-818.)

siRNA bound by a molecular entity is refractory to SYBR Green II (Invitrogen) dye intercalation, resulting in a reduction of fluorescence intensity. The dye exclusion assay monitors this reduction as a function of the increasing molecular entity concentration. Molecular entity-siRNA complexes were prepared in TE buffer by titrating siRNA with increasing amounts of the molecular entity in Greiner Bio-One black 96-well plates. Final concentrations were 10 nM siRNA and 17 pM-1 μM TCPC in a final volume of 100 μl. Binding was allowed to equilibrate for 20 minutes before the addition of 10 μl of a 1:8000 SYBR Green II dilution in TE buffer. Fluorescence was measured using a SpectraMax M5 fluorometer (Molecular Devices) by exciting at 254 nm while monitoring emission at 520 nm. Relative affinities were obtained from resulting binding curves analyzed using GraphPad Prism software.

### Example 26

### Luciferase Knockdown Assay

Human Embryonic Kidney cells (HEK-293) were obtained from the American Type Culture Collection (Mannasas, Va.) and grown in DMEM medium supplemented with 10% fetal bovine serum. Luciferase expressing clones of HEK-293 were generated by transfection with the luciferase mammalian expression vector pGL4 (Promega corp., Madison, Wis.) and drug selected on 500 uG/ml of neomycin. The selected pool was then single cell cloned by limiting dilution. Luciferase expression of individual clone was determined using the Steady Glo assay kit (Promega corporation). A high expression clone, #11, was selected for use in knockdown assays.

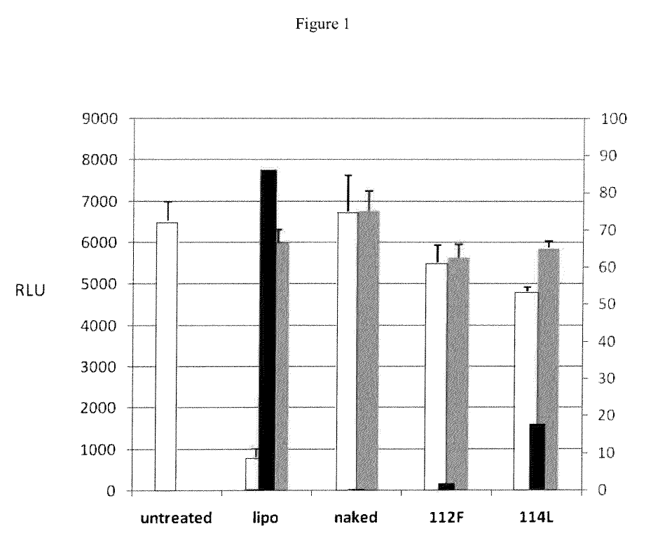

The siRNA sequence encoding siRNA knockdown sequence (SEQ ID No. 1: CCUACGCCGAGUACUUCGACU (sense) and SEQ ID No. 2: UCGAAGUACUCGGCGUAGGUA (antisense)) for luciferase mRNA were purchase from Integrated DNA technologies (San Diego, Calif.). The siRNAs were annealed at 65 degrees for 5 minutes and allowed to cool to room temperature to form 19 bp duplexes with 2 bp overhangs. Control siRNAs using scrambled luciferase knockdown sequence were also obtained from integrated DNA technologies for use as a negative control. For knock down assays, HEK 293-luciferase clone 11 cells were plated at a density of 5000 cells per well in 96 well white assay plates with clear bottoms (corning costar) in 100 ul growth medium per well. For positive control wells, 25 pmol per well of luciferase knockdown siRNA was complexed with lipofectamine 2000 (Invitrogen corp., San Diego, Calif.) as per manufacturer's recommendations. Negative control wells received equals amounts of scrambled sequence complexed with lipofectamine 2000. Test wells received 25 pmols luciferase knockdown siRNA or scrambled siRNA complexed with 125 pmols of test compound diluted in 50 uL of DMEM medium to yield a final test volume of 150 μl per well. After a 72 h incubation of HEK-luciferase cells with test complexes in a 5% CO₂, 37 degree incubator, luciferase expression was measured in a plate luminometer (Molecular Devices M5) using the steady glo luciferase assay kit as per manufacturers recommendations. Percent knockdown was calculated by comparing the luciferase expression of the test compound complexed with the luciferase knockdown sequence versus the luciferase expression of the test compound complexed with the scrambled knockdown sequence. The results is shown in FIG. 1.

siRNA binding, internalization and the luciferase knockdown for the exemplary compounds are scored and listed in Table 1.

TABLE 1
Compound Binding, Internalization and Knock Down Score

| Compound No. | Binding Affinity | Internalization | Knockdown |
|---|---|---|---|
| 9 | − | | |
| 29b | −/+ | | |
| 29c | + | | |
| 31c | ++ | | |
| 31e | + | | |
| 31h | − | | |
| 31i | − | | |
| 31j | ++ | | |
| 31k | + | | |
| 31l | + | | |
| 31m | ++ | | |
| 31n | + | | |
| 31o | ++ | | |
| 31p | + | | |
| 31q | + | | |
| 31r | + | | |
| 31s | + | | |
| 31w | + | | |
| 31y | + | | |
| 2 | + | | |
| 3 | + | | |
| 4 | +++ | | |
| 5 | ++ | | |
| 6 | + | | |

The results show that invention molecular entities are capable of binding to an anionic charged molecule and can be used to deliver charged molecule.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope. Changes therein and other uses will occur to those skilled in the art, which are encompassed within the spirit of the invention, are defined by the scope of the claims.

Definitions provided herein are not intended to be limiting from the meaning commonly understood by one of skill in the art unless indicated otherwise.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A molecular entity comprising an amphiphilic core and at least two charged arms covalently attached thereto, wherein said entity binds, stabilizes and/or facilitates cellular delivery of an opposite charged molecule.

2. The molecular entity of claim 1, wherein said charged arms are positively charged arms and wherein said charged molecule is an anionic charged molecule.

3. The molecular entity of claim 1, wherein one or both of said charged arms further comprise neutral and/or polar functional groups.

4. The molecular entity of claim 2, wherein said positively charged arms comprise a plurality of residues selected from amines, guanidines, amidines, N-containing heterocycles, or combinations thereof.

5. The molecular entity of claim 2, wherein said anionic charged molecule is selected from the group consisting of double-stranded nucleic acid, hairpin nucleic acid, single-stranded DNA, double-stranded DNA, single-stranded RNA, double-stranded RNA and oligonucleotide comprising non-natural monomers.

6. The molecular entity of claim 1, wherein said charged arms are represented by formula I: 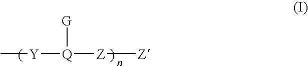
wherein
G is hydrogen, cationically or anionically functionalized side chain;
Y is independently a covalent bond, O, NR¹, C(═X) or S(═O)ₘ,
Z is independently a covalent bond, O, NR¹, C(═X) or S(═O)ₘ,
Q is independently selected from the group consisting of (CH)ₚ, ethylene imine, ethylene glycol and monosaccharide;
Z′ is R¹, OR¹, NR¹ or SR¹;
R¹ is hydrogen or lower alkyl;
X is O, S or NR¹;
n is an integer ranging from 3 to 50;
m is 0, 1, or 2; and
p is 1, 2, 3, or 4.

7. The molecular entity of claim 6, wherein the length of said functionalized side chain is about 3 to about 12 Angstroms.

8. The molecular entity of claim 1, further comprising a bio-recognition molecule.

9. The molecular entity of claim 1, wherein said amphiphilic core comprises at least two attachment sites separated by a distance in the range of about 10 to about 35 Angstroms for linkage of said arms to said core.

10. The molecular entity of claim 9, wherein said core is a linear extended structure.

11. The molecular entity of claim 10, wherein said linear extended structure is a biphenyl derivative.

12. The molecular entity of claim 9, wherein said core is a macrocyclic molecule.

13. The molecular entity of claim 12, wherein said macrocyclic molecule comprises cyclic peptide, cyclic oligosaccharide or cyclic oligoethyleneglycol, provided said cyclic oligosaccharide is not a cyclodextrin.

14. The molecular entity of claim 1, wherein the length of the contiguous backbone of each of said arms is about 12 to about 200 Angstroms.

15. A complex comprising a molecular entity of claim 1 associated with an charged molecule.

16. A composition comprising a pharmaceutical excipient, a charged molecule and a molecular entity of claim 1, or a pharmaceutically acceptable ester, salt, or hydrate thereof.

17. A method for delivering a charged molecule to a cell, said method comprising:
a) binding non-covalently a molecular entity of claim 1 to said charged molecule to form a complex; and
b) contacting said cell with said complex; wherein said charged molecule is taken up by said cell.

18. A method for delivering a charged molecule to a cell, said method comprising contacting said cell with a complex prepared by binding non-covalently a molecular entity of claim 1 to said charged molecule, wherein said charged molecule is taken up by said cell.

19. A method for stabilizing a charged molecule in vivo or for reducing the susceptibility of charged molecules to self-aggregation, said method comprising contacting said charged molecule with a molecular entity of claim 1.

20. A method for (a) increasing the temperature of hybrid dissociation of a double-stranded or hairpin nucleic acid, (b) reducing the susceptibility of a double-stranded or hairpin nucleic acid to digestion by enzymatic nuclease, or (c) reducing the susceptibility of a double-stranded or hairpin nucleic acid to hydrolysis of the phosphodiester backbone, said method comprising contacting said nucleic acid with a molecular entity of claim 5.


SEQUENCE LISTING (21 sequences)

SEQ ID NO: 1 - RNA, 21 nt, Artificial Sequence
  Description of Artificial Sequence Synthetic oligonucleotide
ccuacgccgaguacuucgacu

SEQ ID NO: 2 - RNA, 21 nt, Artificial Sequence
  Description of Artificial Sequence Synthetic oligonucleotide
ucgaaguacucggcguaggua

SEQ ID NO: 3 - protein, 4 aa, Artificial Sequence
  Description of Artificial Sequence Synthetic peptide
GKKK

SEQ ID NO: 4 - protein, 5 aa, Artificial Sequence
  Description of Artificial Sequence Synthetic peptide
AGKKK

SEQ ID NO: 5 - protein, 6 aa, Artificial Sequence
  Description of Artificial Sequence Synthetic peptide
AGGKKK

SEQ ID NO: 6 - protein, 7 aa, Artificial Sequence
  Description of Artificial Sequence Synthetic peptide
GGKKKKK

SEQ ID NO: 7 - protein, 5 aa, Artificial Sequence
  Description of Artificial Sequence Synthetic peptide
KKKKK

SEQ ID NO: 8 - protein, 12 aa, Artificial Sequence
  Description of Artificial Sequence Synthetic peptide
GGKKKKKKKKKK

SEQ ID NO: 9 - protein, 12 aa, Artificial Sequence
  Description of Artificial Sequence Synthetic peptide
CYGRKKRRQRRR

SEQ ID NO: 10 - protein, 5 aa, Artificial Sequence
  Description of Artificial Sequence Synthetic peptide
GKKKK

SEQ ID NO: 11 - protein, 6 aa, Artificial Sequence
  Description of Artificial Sequence Synthetic peptide
GGKKKK

SEQ ID NO: 12 - protein, 6 aa, Artificial Sequence
  Description of Artificial Sequence Synthetic peptide
AGKKKK

SEQ ID NO: 13 - protein, 4 aa, Artificial Sequence
  Description of Artificial Sequence Synthetic peptide
GRRR

SEQ ID NO: 14 - protein, 8 aa, Artificial Sequence
  Description of Artificial Sequence Synthetic peptide
GRRRGKKK

SEQ ID NO: 15 - protein, 4 aa, Artificial Sequence
  Description of Artificial Sequence Synthetic peptide
KKKG

SEQ ID NO: 16 - protein, 5 aa, Artificial Sequence
  Description of Artificial Sequence Synthetic peptide
KKKGG

SEQ ID NO: 17 - protein, 5 aa, Artificial Sequence
  Description of Artificial Sequence Synthetic peptide
KKKKG

SEQ ID NO: 18 - protein, 4 aa, Artificial Sequence
  Description of Artificial Sequence Synthetic peptide
RRRG

SEQ ID NO: 19 - protein, 8 aa, Artificial Sequence
  Description of Artificial Sequence Synthetic peptide
GRRRGKKK

SEQ ID NO: 20 - protein, 4 aa, Artificial Sequence
  Description of Artificial Sequence Synthetic peptide
GKKK

SEQ ID NO: 21 - protein, 9 aa, Artificial Sequence
  Description of Artificial Sequence Synthetic peptide
KLKLKLKLK